(19) United States
(12) Patent Application Publication
(10) Pub. No.: US 2004/0019001 A1
(43) Pub. Date: Jan. 29, 2004

(54) RNA interference mediated inhibition of protein typrosine phosphatase-1B (PTP-1B) gene expression using short interfering RNA

(76) Inventor: James A. McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/206,705

(22) Filed: Jul. 26, 2002

(51) Int. Cl. A61K 48/00; C07H 21/02

(52) U.S. Cl. 514/44; 536/23.1; 435/375

(57) ABSTRACT

The present invention concerns methods and reagents useful in modulating gene expression in a variety of applications, including use in therapeutic, diagnostic, target validation, and genomic discovery applications associated with insulin response. Specifically, the invention relates to small interfering RNA (siRNA) molecules capable of mediating RNA interference (RNAi) against PTP-1B polypeptide and polynucleotide targets.

### BACKGROUND Of The INVENTION

The present invention concerns methods and reagents useful in modulating protein tyrosine phosphatase-1B (PTP-1B) gene expression in a variety of applications, including use in therapeutic, diagnostic, target validation, and genomic discovery applications. Specifically, the invention relates to short interfering nucleic acid molecules capable of mediating RNA interference (RNAi) PTP-1B expression.

The following is a discussion of relevant art pertaining to RNAi. The discussion is provided only for understanding of the invention that follows. The summary is not an admission that any of the work described below is prior art to the claimed invention.

RNA interference refers to the process of sequence-specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877) has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO00/44914, and Beach et al., International PCT Publication No. WO01/68836 both suggest that siRNA “may include modifications to either the phosphate-sugar back bone or the nucleoside to include at least one of a nitrogen or sulfur heteroatom”, however neither application teaches to what extent these modifications are tolerated in siRNA molecules nor provide any examples of such modified siRNA. Kreutzer and Limmer, Canadian Patent Application No. 2,359,180, also describe certain chemical modifications for use in dsRNA constructs in order to counteract activation of double stranded-RNA-dependent protein kinase PKR, specifically 2′-amino or 2′-O-methyl nucleotides, and nucleotides containing a 2′-O or 4′-C methylene bridge. However, Kreutzer and Limmer similarly fail to show to what extent these modifications are tolerated in siRNA molecules nor do they provide any examples of such modified siRNA.

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that “RNAs with two [phosphorothioate] modified bases also had substantial decreases in effectiveness as RNAi triggers (data not shown); [phosphorothioate] modification of more than two residues greatly destabilized the RNAs in vitro and we were not able to assay interference activities.” Id. at 1081. The authors also tested certain modifications at the 2′-position of the nucleotide sugar in the long siRNA transcripts and observed that substituting deoxynucleotides for ribonucleotides “produced a substantial decrease in interference activity”, especially in the case of Uridine to Thymidine and/or Cytidine to deoxy-Cytidine substitutions. Id. In addition, the authors tested certain base modifications, including substituting 4-thiouracil, 5-bromouracil, 5-iodouracil, 3-(aminoallyl)uracil for uracil, and inosine for guanosine in sense and antisense strands of the siRNA, and found that whereas 4-thiouracil and 5-bromouracil were all well tolerated, inosine “produced a substantial decrease in interference activity” when incorporated in either strand. Incorporation of 5-iodouracil and 3-(aminoallyl)uracil in the antisense strand resulted in substantial decrease in RNAi activity as well.

Beach et al., International PCT Publication No. WO01/68836, describes specific methods for attenuating gene expression using endogenously derived dsRNA. Tuschl et al., International PCT Publication No. WO01/75164, describes a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due “to the danger of activating interferon response”. Li et al., International PCT Publication No. WO00/44914, describes the use of specific dsRNAs for use in attenuating the expression of certain target genes. Zernicka-Goetz et al., International PCT Publication No. WO 01/36646, describes certain methods for inhibiting the expression of particular genes in mammalian cells using certain dsRNA molecules. Fire et al., International PCT Publication No. WO99/32619, describes particular methods for introducing certain dsRNA molecules into cells for use in inhibiting gene expression. Plaetinck et al., International PCT Publication No. WO00/01846, describes certain methods for identifying specific genes responsible for conferring a particular phenotype in a cell using specific dsRNA molecules. Mello et al., International PCT Publication No. WO01/29058, describes the identification of specific genes involved in dsRNA mediated RNAi. Deschamps Depaillette et al., International PCT Publication No. WO99/07409, describes specific compositions consisting of particular dsRNA molecules combined with certain anti-viral agents. Driscoll et al., International PCT Publication No. WO01/49844, describes specific DNA constructs for use in facilitating gene silencing in targeted organisms. Parrish et al., 2000, Molecular Cell, 6, 1977-1087, describes specific chemically modified siRNA constructs targeting the unc-22 gene of C. elegans. Tuschl et al., International PCT Publication No. WO02/44321, describe certain synthetic siRNA constructs.

Protein tyrosine phosphorylation and dephosphorylation are important mechanisms in the regulation of signal transduction pathways that control the processes of cell growth, proliferation, and differentiation (Fantl, W. J., 1993, Annu. Rev. Biochem., 62, 453-481). Cooperative enzyme classes regulate protein tyrosine phosphorylation and dephosphorylation events. These broad classes of enzymes consist of the protein tyrosine kinases (PTKs) and protein tyrosine phosphatases (PTPs). PTKs and PTPs can exist as both receptor-type transmembrane proteins and as cytoplasmic protein enzymes. Receptor tyrosine kinases propagate signal transduction events via extracellular receptor-ligand interactions that result in the activation of the tyrosine kinase portion of the PTK in the cytoplasmic domain. Receptor-like transmembrane PTPs function through extracellular ligand binding that modulates dephosphorylation of intracellular phosphotyrosine proteins via cytoplasmic phosphatase domains. Cytoplasmic PTKs and PTPs exert enzymatic activity without receptor-mediated ligand interactions, however, phosphorylation can regulate the activity of these enzymes.

Protein tyrosine phosphatase 1B, a cytoplasmic PTP, was the first PTP to be isolated in homogeneous form (Tonks, N. K., 1988, J. Biol. Chem., 263, 6722-6730), characterized (Tonks, N. K., 1988, J. Biol. Chem., 263, 6731-6737), and sequenced (Charbonneau, H., 1989, Biochemistry, 86, 5252-5256). Cytoplasmic and receptor-like PTPs both share a catalytic domain characterized by eleven conserved amino acids containing cysteine and arginine residues that are critical for phosphatase activity (Streuli, M., 1990, EMBO, 9, 2399-2407). A cysteine residue at position 215 is responsible for the covalent attachment of phosphate to the enzyme (Guan, K., 1991, J. Biol. Chem., 266, 17026-17030). The crystal structure of human PTP1B defined the phosphate binding site of the enzyme as a glycine rich cleft at the surface of the molecule with cysteine 215 positioned at the base of this cleft. The location of cysteine 215 and the shape of the cleft provide specificity of PTPase activity for tyrosine residues but not for serine or threonine residues (Barford, D., 1994, Science, 263, 1397-1404).

Receptor tyrosine kinase and protein tyrosine phosphatase localization plays a key role in the regulation of phosphotyrosine mediated signal transduction. PTP-1B activity and specificity against a panel of receptor tyrosine kinases demonstrated clear differences between substrates, suggesting that cellular compartmentalization is a determinant in defining the activity and function of the enzyme (Lammers, R., 1993, J. Biol. Chem., 268, 22456-22462). Experiments have indicated that PTP-1B is localized predominantly in the endoplasmic reticulum via its 35 amino acid carboxyterminal sequence. PTP-1B is also tightly associated with microsomal membranes with its catalytic phosphatase domain oriented towards the cytoplasm (Frangioni, J. V., 1992, Cell, 68, 545-560).

PTP-1B has been identified as a negative regulator of the insulin response. PTP-1B is widely expressed in insulin sensitive tissues (Goldstein, B. J., 1993, Receptor, 3, 1-15). Isolated PTP-1B dephosphorylates the insulin receptor in vitro (Tonks, N. K., 1988, J. Biol. Chem., 263, 6731-6737). PTP-1B dephosphorylation of multiple phosphotyrosine residues of the insulin receptor proceeds sequentially and with specificity for the three tyrosine residues that are critical for receptor autoactivation (Ramachandran, C., 1992, Biochemistry, 31, 4232-4238). In addition to insulin receptor dephosphorylation, PTP-1B also dephosphorylates the insulin related substrate 1 (IRS-1), a principal substrate of the insulin receptor (Lammers, R., 1993, J. Biol. Chem., 268, 22456-22462).

Microinjection of PTP1B into Xenopus oocytes results in the inhibition of insulin stimulated tyrosine phosphorylation of endogenous proteins, including the beta-subunit of the insulin and insulin-like growth factor receptor proteins. The resulting 3 to 5 fold increase over endogenous PTPase activity also blocks the activation of an S6 peptide kinase (Cicirelli, M. F., 1990, Proc, Natl. Acad. Sci., 87, 5514-5518). Inactivation of recombinant rat PTP-1B with antibody immunoprecipitation results in the dramatic increase in insulin stimulated DNA synthesis and phosphatidylinositol 3′-kinase activity. Insulin stimulated receptor autophosphorylation and insulin receptor substrate 1 tyrosine phosphorylation are increased dramatically as well through PTP-1B inhibition (Ahmad, F., 1995, J. Biol. Chem., 270, 20503-20508).

Increased PTP-1B expression correlates with insulin resistance in hyperglycemic cultured fibroblasts. In this study, desensitized insulin receptor function was observed via impaired insulin-induced autophosphorylation of the receptor. Treatment with insulin sensitivity normalizing thiazolidine derivatives resulted in the amelioration of the hyperglycemic insulin resistance via a normalization in PTP-1B expression (Maegawa, H., 1995, J. Biol. Chem., 270, 7724-7730). A murine model of insulin resistance with a knockout of the hetrerotrimeric GTP-binding protein subunit Gi-alpha-2 provides a type 2 diabetes phenotype that correlates with the increased expression of PTP-1B (Moxam, C. M., 1996, Nature, 379, 840-844).

PTP-1B interacts directly with the activated insulin receptor beta-subunit. An inactive homolog of PTP-1B was used to precipitate the activated insulin receptor in both purified receptor preparations and whole-cell lysates. Phosphorylation of the insulin receptor's triple tyrosine residues in the kinase domain is necessary for PTP-1B interaction. Furthermore, insulin stimulates tyrosine phosphorylation of PTP-1B (Seely, B. L., 1996, Diabetes, 45, 1379-1385). A similar study confirmed the direct interaction of PTP-1B with the insulin receptor beta-subunit as well as the required multiple phosphorylation sites within the receptor and PTP-1B (Bandyopadhyay, D., J. Biol. Chem., 272, 1639-1645).

Knockout mice lacking the PTP-1B gene (both homozygous PTP-1B^{-/-} and heterozygous PTP-1B^{+/-}) have been used to study the specific role of PTP-1B relating to insulin action in vivo. The resulting PTP-1B deficient mice were healthy and, in the fed state, had lower blood glucose and circulating insulin levels that were half that of their PTP-1B^{+/+} expressing littermates. These PTP-1B deficient mice demonstrated enhanced insulin sensitivity in glucose and insulin tolerance tests. At the physiological level, the PTP-1B deficient mice showed increased phosphorylation of the insulin receptor after insulin administration. When fed a high fat diet, the PTP-1B deficient mice were resistant to weight gain and remained insulin sensitive as opposed to normal PTP-1B expressing mice, who rapidly gained weight and become insulin resistant (Elchebly, M., 1999, Science, 283, 1544-1548). As such, modulation of PTP-1B expression could be used to regulate autophosphorylation of the insulin receptor and increase insulin sensitivity in vivo. This modulation could prove beneficial in the treatment of insulin related disease states.

In light of the above findings, particular disease states that involve PTP-1B expression include but are not limited to:

1. Diabetes: Both type 1 and type 2 diabetes can be treated by modulation of PTP-1B expression. Type 2 diabetes correlates to desensitized insulin receptor function (White et al., 1994). Disruption of the PTP-1B dephosphorylation of the insulin receptor in vivo manifests in insulin sensitivity and increased insulin receptor autophosphorylation (Elchebly et al., 1999). Insulin dependant diabetes, type 1, can respond to PTP-1B modulation through increased insulin sensitivity.

2. Obesity: Elchebly et al., 1999, demonstrated that PTP-1B deficient mice were resistant to weight gain when fed a high fat diet compared to normal PTP-1B expressing mice. This finding suggests that PTP-1B modulation can be beneficial in the treatment of obesity. Ahmad et al., 1997, Metab. Clin. Exp., 46, 1140-1145, describe reduced PTPs in adipose tissue and improved insulin sensitivity in obese subjects following weight loss.

The human genome is thought to contain up to 100 PTPases, each varying slightly in chemistry but vastly in function. Compounds designed to inhibit PTP-1B activity specifically by covalent binding to or modification of PTP-1B have the potential for multiple side effects. Conventional drug substances that will potently suppress PTP-1B activity with few or no side effects from interaction with other PTPs are difficult to envision. A more attractive approach to PTP-1B modulation would involve the specific regulation of PTP-1B expression with nucleic acid technologies such as siRNA mediated RNAi.

MsSwiggen et al., International PCT Publication No. WO01/16312, describes nucleic acid modulators of PTP-1B.

### SUMMARY OF THE INVENTION

One embodiment of the invention provides a short interfering RNA (siRNA) molecule that down regulates expression of a protein tyrosine phosphatase-1B (PTP-1B) gene by RNA interference. The siRNA molecule can be adapted for use to treat type I diabetes, type II diabetes, obesity or a combination thereof. The siRNA molecule can comprise a sense region and an antisense region. The antisense region can comprise sequence complementary to an RNA sequence encoding PTP-1B and the sense region can comprise sequence complementary to the antisense region.

The siRNA molecule can be assembled from two nucleic acid fragments wherein one fragment comprises the sense region and the second fragment comprises the antisense region of said siRNA molecule. The sense region and antisense region can be covalently connected via a linker molecule. The linker molecule can be a polynucleotide linker or a non-nucleotide linker.

The antisense region can comprise a sequence complementary to sequence having any of SEQ ID NOs. 1-185. The antisense region can also comprise sequence having any of SEQ ID NOs. 186-370, 384, 386, 388, 390, 392, or 394. The sense region can comprise sequence having any of SEQ ID NOs. 1-185, 383, 385, 387, 389, 391, or 393. The sense region can comprise a sequence of SEQ ID NO. 371 and the antisense region can comprise a sequence of SEQ ID NO. 372. The sense region can comprise a sequence of SEQ ID NO. 373 and the antisense region can comprise a sequence of SEQ ID NO. 374. The sense region can comprise a sequence of SEQ ID NO. 375 and the antisense region can comprise a sequence of SEQ ID NO. 376. The sense region can comprise a sequence of SEQ ID NO. 377 and the antisense region can comprise a sequence of SEQ ID NO. 378. The sense region can comprise a sequence of SEQ ID NO. 379 and the antisense region can comprise a sequence of SEQ ID NO. 380. The sense region can comprise a sequence of SEQ ID NO. 381 and the antisense region can comprise a sequence of SEQ ID NO. 382.

The sense region of a siRNA molecule of the invention can comprise a 3′-terminal overhang and the antisense region can comprise a 3′-terminal overhang. The 3′-terminal overhangs each can comprise about 2 nucleotides. The antisense region of the 3′-terminal nucleotide overhang can be complementary to RNA encoding PTP-1B.

The sense region of a siRNA molecule can comprise one or more 2′-O-methyl modified pyrimidine nucleotides. The sense region can comprise a terminal cap moiety at the 5′-end, 3′-end, or both 5′ and 3′ ends of said sense region.

The antisense region of a siRNA molecule can comprise one or more 2′-deoxy-2′-fluoro modified pyrimidine nucleotides. The antisense region can also comprise a phosphorothioate internucleotide linkage at the 3′ end of said antisense region. The antisense region can comprise between about one and about five phosphorothioate internucleotide linkages at the 5′ end of said antisense region.

The 3′-terminal nucleotide overhangs of a siRNA molecule can comprise ribonucleotides or deoxyribonucleotides that are chemically modified at a nucleic acid sugar, base, or backbone. The 3′-terminal nucleotide overhangs can also comprise one or more universal base ribonucleotides. Additionally, the 3′-terminal nucleotide overhangs can comprise one or more acyclic nucleotides.

The 3′-terminal nucleotide overhangs can comprise nucleotides comprising internucleotide linkages having Formula I:

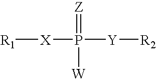

wherein each R1 and R2 is independently any nucleotide, non-nucleotide, or polynucleotide which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all O.

The 3′-terminal nucleotide overhangs can comprise nucleotides or non-nucleotides having Formula II:

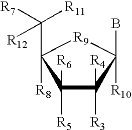

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base or any other non-naturally occurring base that can be complementary or non-complementary to PTP-1B RNA or a non-nucleosidic base or any other non-naturally occurring universal base that can be complementary or non-complementary to PTP-1B RNA.

Another embodiment of the invention provides an expression vector comprising a nucleic acid sequence encoding at least one siRNA molecule of the invention in a manner that allows expression of the nucleic acid molecule. The expression vector can be in a mammalian cell, such as a human cell. The siRNA molecule can comprise a sense region and an antisense region. The antisense region can comprise sequence complementary to an RNA sequence encoding PTP-1B and the sense region comprises sequence complementary to the antisense region. The siRNA molecule can comprise two distinct strands having complementarity sense and antisense regions or can comprise a single strand having complementary sense and antisense regions.

Therefore, this invention relates to compounds, compositions, and methods useful for modulating gene expression, for example, genes associated with insulin signalling, such as diabetes and obesity, by RNA interference (RNAi) using short interfering RNA (siRNA). In particular, the instant invention features siRNA molecules and methods to modulate the expression of PTP-1B. The siRNA of the invention can be unmodified or chemically modified. The siRNA of the instant invention can be chemically synthesized, expressed from a vector or enzymatically synthesized. The instant invention also features various chemically modified synthetic short interfering RNA (siRNA) molecules capable of modulating PTP-1B gene expression/activity in cells by RNA inference (RNAi). The use of chemically modified siRNA is expected to improve various properties of native siRNA molecules through increased resistance to nuclease degradation in vivo and/or improved cellular uptake. The siRNA molecules of the instant invention provide useful reagents and methods for a variety of therapeutic, diagnostic, agricultural, target validation, genomic discovery, genetic engineering and pharmacogenomic applications.

In one embodiment, the invention features one or more siRNA molecules and methods that independently or in combination modulate the expression of gene(s) encoding proteins associated with insulin signalling disorders or conditions such as diabetes (type I and type II), and obesity. Specifically, the present invention features siRNA molecules that modulate the expression of proteins associated insulin response and related pathologies, for example PTP-1B (Genbank Accession No NM_{—}002827).

The description below of the various aspects and embodiments is provided with reference to the exemplary PTP-1B gene/protein, including components or subunits thereof. However, the various aspects and embodiments are also directed to other genes which express other PTP-1B related proteins or other proteins associated with insulin response. Those additional genes can be analyzed for target sites using the methods described for PTP-1B herein. Thus, the inhibition and the effects of such inhibition of the other genes can be performed as described herein.

In one embodiment, the invention features a siRNA molecule which down regulates expression of a PTP-1B gene, for example, wherein the PTP-1B gene comprises PTP-1B encoding sequence.

In one embodiment, the invention features a siRNA molecule having RNAi activity against PTP-1B RNA, wherein the siRNA molecule comprises a sequence complementary to any RNA having PTP-1B encoding sequence, for example Genbank Accession No. NM_{—}002827.

In another embodiment, the invention features a siRNA molecule comprising sequences selected from the group consisting of SEQ ID NOs: 1-370. In another embodiment, the invention features a siRNA molecule having an antisense region complementary to any sequence having SEQ ID NOs: 1-185. In another embodiment, the invention features a siRNA molecule having an antisense region having any of SEQ ID NOs: 186-370. In another embodiment, the invention features a siRNA molecule having an antisense region having any of SEQ ID NOs: 1-185. In yet another embodiment, the invention features a siRNA molecule comprising a sequence, for example the antisense sequence of the siRNA construct, complementary to a sequence or portion of sequence comprising Genbank Accession No. NM_{—}002827 (PTP-1B).

In one embodiment, a siRNA molecule of the invention has RNAi activity that modulates expression of RNA encoded by a PTP-1B gene.

In one embodiment, nucleic acid molecules of the invention that act as mediators of the RNA interference gene silencing response are double stranded RNA molecules. In another embodiment, the siRNA molecules of the invention consist of duplexes containing about 19 base pairs between oligonucleotides comprising about 19 to about 25 nucleotides (e.g., about 19, 20, 21, 22, 23, 24, or 25). In yet another embodiment, siRNA molecules of the invention comprise duplexes with overhanging ends of 1-3 (e.g., 1, 2, or 3) nucleotides, for example 21 nucleotide duplexes with 19 base pairs and 2 nucleotide 3′-overhangs. These nucleotide overhangs in the antisense strand are optionally complementary to the target sequence.

In one embodiment, the invention features chemically modified siRNA constructs having specificity for PTP-1B expressing nucleic acid molecules. Non-limiting examples of such chemical modifications include without limitation phosphorothioate internucleotide linkages, 2′-O-methyl ribonucleotides, 2′-deoxy-2′-fluoro ribonucleotides, “universal base” nucleotides, 5-C-methyl nucleotides, and inverted deoxyabasic residue incorporation. These chemical modifications, when used in various siRNA constructs, are shown to preserve RNAi activity in cells while at the same time, dramatically increasing the serum stability of these compounds. Furthermore, contrary to the data published by Parrish et al., supra, applicant demonstrates that multiple (greater than one) phosphorothioate substitutions are well tolerated and confer substantial increases in serum stability for modified siRNA constructs. Chemical modifications of the siRNA constructs can also be used to improve the stability of the interaction with the target RNA sequence and to improve nuclease resistance.

In a non-limiting example, the introduction of chemically modified nucleotides into nucleic acid molecules will provide a powerful tool in overcoming potential limitations of in vivo stability and bioavailability inherent to native RNA molecules that are delivered exogenously. For example, the use of chemically modified nucleic acid molecules can enable a lower dose of a particular nucleic acid molecule for a given therapeutic effect since chemically modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically modified nucleic acid molecule is reduced as compared to a native nucleic acid molecule, for example when compared to an all RNA nucleic acid molecule, the overall activity of the modified nucleic acid molecule can be greater than the native molecule due to improved stability and/or delivery of the molecule. Unlike native unmodified siRNA, chemically modified siRNA can also minimize the possibility of activating interferon activity in humans.

In one embodiment, the invention features a chemically modified short interfering RNA (siRNA) molecule capable of mediating RNA interference (RNAi) against PTP-1B inside a cell, wherein the chemical modification comprises one or more (e.g., about 1, 2, 3, 4, 5, 6, 7, 8, 9, 10, or more) nucleotides comprising a backbone modified internucleotide linkage having Formula I:

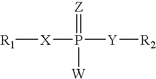

wherein each R1 and R2 is independently any nucleotide, non-nucleotide, or polynucleotide which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all O.

The chemically modified internucleotide linkages having Formula I, for example wherein any Z, W, X, and/or Y independently comprises a sulphur atom, can be present in one or both oligonucleotide strands of the siRNA duplex, for example in the sense strand, antisense strand, or both strands. The siRNA molecules of the invention can comprise one or more (e.g., about 1, 2, 3, 4, 5, 6, 7, 8, 9, 10, or more) chemically modified internucleotide linkages having Formula I at the 3′-end, 5′-end, or both 3′ and 5′-ends of the sense strand, antisense strand, or both strands. For example, an exemplary siRNA molecule of the invention can comprise between about 1 and about 5 or more (e.g., about 1, 2, 3, 4, 5, or more) chemically modified internucleotide linkages having Formula I at the 5′-end of the sense strand, antisense strand, or both strands. In another non-limiting example, an exemplary siRNA molecule of the invention can comprise one or more (e.g., about 1, 2, 3, 4, 5, 6, 7, 8, 9, 10, or more) pyrimidine nucleotides with chemically modified internucleotide linkages having Formula I in the sense strand, antisense strand, or both strands. In yet another non-limiting example, an exemplary siRNA molecule of the invention can comprise one or more (e.g., about 1, 2, 3, 4, 5, 6, 7, 8, 9, 10, or more) purine nucleotides with chemically modified internucleotide linkages having Formula I in the sense strand, antisense strand, or both strands. In another embodiment, a siRNA molecule of the invention having internucleotide linkage(s) of Formula I also comprises a chemically modified nucleotide or non-nucleotide having any of Formulae II, III, V, or VI.

In one embodiment, the invention features a chemically modified short interfering RNA (siRNA) molecule capable of mediating RNA interference (RNAi) against PTP-1B inside a cell, wherein the chemical modification comprises one or more (e.g., about 1, 2, 3, 4, 5, 6, 7, 8, 9, 10, or more) nucleotides or non-nucleotides having Formula II:

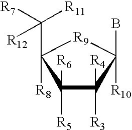

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to be complementary or non-complementary to RNA or a non-nucleosidic base such as phenyl, naphthyl, 3-nitropyrrole, 5-nitroindole, nebularine, pyridone, pyridinone, or any other non-naturally occurring universal base that can be employed to be complementary or non-complementary to RNA.

The chemically modified nucleotide or non-nucleotide of Formula II can be present in one or both oligonucleotide strands of the siRNA duplex, for example in the sense strand, antisense strand, or both strands. The siRNA molecules of the invention can comprise one or more chemically modified nucleotide or non-nucleotide of Formula II at the 3′-end, 5′-end, or both 3′ and 5′-ends of the sense strand, antisense strand, or both strands. For example, an exemplary siRNA molecule of the invention can comprise between about 1 and about 5 or more (e.g., about 1, 2, 3, 4, 5, or more) chemically modified nucleotide or non-nucleotide of Formula II at the 5′-end of the sense strand, antisense strand, or both strands. In anther non-limiting example, an exemplary siRNA molecule of the invention can comprise between about 1 and about 5 or more (e.g., about 1, 2, 3, 4, 5, or more) chemically modified nucleotide or non-nucleotide of Formula II at the 3′-end of the sense strand, antisense strand, or both strands.

In one embodiment, the invention features a chemically modified short interfering RNA (siRNA) molecule capable of mediating RNA interference (RNAi) against PTP-1B inside a cell, wherein the chemical modification comprises one or more (e.g., about 1, 2, 3, 4, 5, 6, 7, 8, 9, 10, or more) nucleotides or non-nucleotides having Formula III:

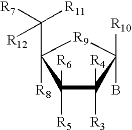

wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base that can be employed to be complementary or non-complementary to RNA or a non-nucleosidic base such as phenyl, naphthyl, 3-nitropyrrole, 5-nitroindole, nebularine, pyridone, pyridinone, or any other non-naturally occurring universal base that can be employed to be complementary or non-complementary to RNA.

The chemically modified nucleotide or non-nucleotide of Formula III can be present in one or both oligonucleotide strands of the siRNA duplex, for example in the sense strand, antisense strand, or both strands. The siRNA molecules of the invention can comprise one or more chemically modified nucleotide or non-nucleotide of Formula III at the 3′-end, 5′-end, or both 3′ and 5′-ends of the sense strand, antisense strand, or both strands. For example, an exemplary siRNA molecule of the invention can comprise between about 1 and about 5 or more (e.g., about 1, 2, 3, 4, 5, or more) chemically modified nucleotide or non-nucleotide of Formula III at the 5′-end of the sense strand, antisense strand, or both strands. In anther non-limiting example, an exemplary siRNA molecule of the invention can comprise between about 1 and about 5 or more (e.g., about 1, 2, 3, 4, 5, or more) chemically modified nucleotide or non-nucleotide of Formula III at the 3′-end of the sense strand, antisense strand, or both strands.

In another embodiment, a siRNA molecule of the invention comprises a nucleotide having Formula II or III, wherein the nucleotide having Formula II or III is in an inverted configuration. For example, the nucleotide having Formula II or III is connected to the siRNA construct in a 3′,3′, 3′-2′, 2′-3′, or 5′,5′ configuration, such as at the 3′-end, 5′-end, or both 3′ and 5′ ends of one or both siRNA strands.

In one embodiment, the invention features a chemically modified short interfering RNA (siRNA) molecule capable of mediating RNA interference (RNAi) against PTP-1B inside a cell, wherein the chemical modification comprises a 5′-terminal phosphate group having Formula IV:

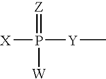

wherein each X and Y is independently O, S, N, alkyl, substituted alkyl, or alkylhalo; each Z and W is independently O, S, N, alkyl, substituted alkyl, O-alkyl, S-alkyl, alkaryl, aralkyl, or alkylhalo; and wherein W, X, Y and Z are not all O.

In one embodiment, the invention features a siRNA molecule having a 5′-terminal phosphate group having Formula IV on the target-complementary strand, for example a strand complementary to PTP-1B RNA, wherein the siRNA molecule comprises an all RNA siRNA molecule. In another embodiment, the invention features a siRNA molecule having a 5′-terminal phosphate group having Formula IV on the target-complementary strand wherein the siRNA molecule also comprises 1-3 (e.g., 1, 2, or 3) nucleotide 3′-overhangs having between about 1 and about 4 (e.g., about 1, 2, 3, or 4) deoxyribonucleotides on the 3′-end of one or both strands. In another embodiment, a 5′-terminal phosphate group having Formula IV is present on the target-complementary strand of a siRNA molecule of the invention, for example a siRNA molecule having chemical modifications having Formula I, Formula II and/or Formula III.

In one embodiment, the invention features a chemically modified short interfering RNA (siRNA) molecule capable of mediating RNA interference (RNAi) against PTP-1B inside a cell, wherein the chemical modification comprises one or more phosphorothioate internucleotide linkages. For example, in a non-limiting example, the invention features a chemically modified short interfering RNA (siRNA) having about 1, 2, 3, 4, 5, 6, 7, 8 or more phosphorothioate internucleotide linkages in one siRNA strand. In yet another embodiment, the invention features a chemically modified short interfering RNA (siRNA) individually having about 1, 2, 3, 4, 5, 6, 7, 8 or more phosphorothioate internucleotide linkages in both siRNA strands. The phosphorothioate internucleotide linkages can be present in one or both oligonucleotide strands of the siRNA duplex, for example in the sense strand, antisense strand, or both strands. The siRNA molecules of the invention can comprise one or more phosphorothioate internucleotide linkages at the 3′-end, 5′-end, or both 3′ and 5′-ends of the sense strand, antisense strand, or both strands. For example, an exemplary siRNA molecule of the invention can comprise between about 1 and about 5 or more (e.g., about 1, 2, 3, 4, 5, or more) consecutive phosphorothioate internucleotide linkages at the 5′-end of the sense strand, antisense strand, or both strands. In another non-limiting example, an exemplary siRNA molecule of the invention can comprise one or more (e.g., about 1, 2, 3, 4, 5, 6, 7, 8, 9, 10, or more) pyrimidine phosphorothioate internucleotide linkages in the sense strand, antisense strand, or both strands. In yet another non-limiting example, an exemplary siRNA molecule of the invention can comprise one or more (e.g., about 1, 2, 3, 4, 5, 6, 7, 8, 9, 10, or more) purine phosphorothioate internucleotide linkages in the sense strand, antisense strand, or both strands.

In one embodiment, the invention features a siRNA molecule, wherein the sense strand comprises one or more, for example about 1, 2, 3, 4, 5, 6, 7, 8 , 9 , 10 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5 or more) universal base modified nucleotides, and optionally a terminal cap molecule at the 3′, 5′, or both 3′ and 5′-ends of the sense strand; and wherein the antisense strand comprises any of between 1 and 10 or more, specifically about 1, 2, 3, 4, 5, 6, 7, 8 , 9 , 10 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5 or more) universal base modified nucleotides, and optionally a terminal cap molecule at the 3′, 5′, or both 3′ and 5′-ends of the antisense strand. In another embodiment, one or more, for example about 1, 2, 3, 4, 5, 6, 7, 8, 9, 10 or more pyrimidine nucleotides of the sense and/or antisense siRNA stand are chemically modified with 2′-deoxy, 2′-O-methyl and/or 2′-deoxy-2′-fluoro nucleotides, with or without one or more, for example about 1, 2, 3, 4, 5, 6, 7, 8, 9, 10 or more phosphorothioate internucleotide linkages and/or a terminal cap molecule at the 3′, 5′, or both 3′ and 5′-ends, being present in the same or different strand.

In another embodiment, the invention features a siRNA molecule, wherein the sense strand comprises between about 1 and about 5, specifically about 1, 2, 3, 4, or 5 phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base modified nucleotides, and optionally a terminal cap molecule at the 3′, 5′, or both 3′ and 5′-ends of the sense strand; and wherein the antisense strand comprises any of between about 1 and about 5 or more, specifically about 1, 2, 3, 4, 5, or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5 or more) universal base modified nucleotides, and optionally a terminal cap molecule at the 3′, 5′, or both 3′ and 5′-ends of the antisense strand. In another embodiment, one or more, for example about 1, 2, 3, 4, 5, 6, 7, 8, 9, 10 or more pyrimidine nucleotides of the sense and/or antisense siRNA stand are chemically modified with 2′-deoxy, 2′-O-methyl and/or 2′-deoxy-2′-fluoro nucleotides, with or without between about 1 and about 5 or more, for example about 1, 2, 3, 4, 5 or more phosphorothioate internucleotide linkages and/or a terminal cap molecule at the 3′, 5′, or both 3′ and 5′-ends, being present in the same or different strand.

In one embodiment, the invention features a siRNA molecule, wherein the antisense strand comprises one or more, for example about 1, 2, 3, 4, 5, 6, 7, 8 , 9 , 10 or more phosphorothioate internucleotide linkages, and/or between one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base modified nucleotides, and optionally a terminal cap molecule at the 3′, 5′, or both 3′ and 5′-ends of the sense strand; and wherein the antisense strand comprises any of between about 1 and about 10, specifically about 1, 2, 3, 4, 5, 6, 7, 8 , 9 , 10 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base modified nucleotides, and optionally a terminal cap molecule at the 3′, 5′, or both 3′ and 5′-ends of the antisense strand. In another embodiment, one or more, for example about 1, 2, 3, 4, 5, 6, 7, 8, 9, 10 or more pyrimidine nucleotides of the sense and/or antisense siRNA stand are chemically modified with 2′-deoxy, 2′-O-methyl and/or 2′-deoxy-2′-fluoro nucleotides, with or without one or more, for example about 1, 2, 3, 4, 5, 6, 7, 8, 9, 10 or more phosphorothioate internucleotide linkages and/or a terminal cap molecule at the 3′, 5′, or both 3′ and 5′-ends, being present in the same or different strand.

In another embodiment, the invention features a siRNA molecule, wherein the antisense strand comprises between about 1 and about 5 or more, specifically about 1, 2, 3, 4, 5 or more phosphorothioate internucleotide linkages, and/or one or more (e.g., about 1, 2, 3, 4, 5 or more) 2′-deoxy, 2′-O-methyl, 2′-deoxy-2′-fluoro, and/or one or more (e.g., about 1, 2, 3, 4, 5 or more) universal base modified nucleotides, and optionally a terminal cap molecule at the 3′, 5′, or both 3′ and 5′-ends of the sense strand; and wherein the antisense strand comprises any of between about 1 and about 5 or more, specifically about 1, 2, 3, 4, 5 or more phosphorothioate internucleotide linkages, and/or one or more (e.g., about 1, 2, 3, 4, 5, or more) 2′-deoxy, 2′-O-methyl, 2′-deoxy-2′-fluoro, and/or one or more (e.g., about 1, 2, 3, 4, 5, or more) universal base modified nucleotides, and optionally a terminal cap molecule at the 3′, 5′, or both 3′ and 5′-ends of the antisense strand. In another embodiment, one or more, for example about 1, 2, 3, 4, 5, 6, 7, 8, 9, 10 or more pyrimidine nucleotides of the sense and/or antisense siRNA stand are chemically modified with 2′-deoxy, 2′-O-methyl and/or 2′-deoxy-2′-fluoro nucleotides, with or without between about 1 and about 5, for example about 1, 2, 3, 4, 5 or more phosphorothioate internucleotide linkages and/or a terminal cap molecule at the 3′, 5′, or both 3′ and 5′-ends, being present in the same or different strand.

In one embodiment, the invention features a chemically modified short interfering RNA (siRNA) molecule having between about 1 and about 5, specifically 1, 2, 3, 4, 5 or more phosphorothioate internucleotide linkages in each strand of the siRNA molecule.

In another embodiment, the invention features a siRNA molecule comprising 2′-5′ internucleotide linkages. The 2′-5′ internucleotide linkage(s) can be at the 5′-end, 3′-end, or both 5′ and 3′ ends of one or both siRNA sequence strands. In addition, the 2′-5′ internucleotide linkage(s) can be present at various other positions within one or both siRNA sequence strands, for example, about 1, 2, 3, 4, 5, 6, 7, 8, 9, 10, or more including every internucleotide linkage of a pyrimidine nucleotide in one or both strands of the siRNA molecule can comprise a 2′-5′ internucleotide linkage, or about 1, 2, 3, 4, 5, 6, 7, 8, 9, 10, or more including every internucleotide linkage of a purine nucleotide in one or both strands of the siRNA molecule can comprise a 2′-5′ internucleotide linkage.

In another embodiment, a chemically modified siRNA molecule of the invention comprises a duplex having two strands, one or both of which can be chemically modified, wherein each strand is between about 18 and about 27 (e.g., about 18, 19, 20, 21, 22, 23, 24, 25, 26, or 27) nucleotides in length, wherein the duplex has between about 18 and about 23 (e.g., about 18, 19, 20, 21, 22, or 23) base pairs, and wherein the chemical modification comprises a structure having Formula I, Formula II, Formula III and/or Formula IV. For example, an exemplary chemically modified siRNA molecule of the invention comprises a duplex having two strands, one or both of which can be chemically modified with a chemical modification having Formula I, Formula II, Formula III, and/or Formula IV, wherein each strand consists of 21 nucleotides, each having 2 nucleotide 3′-overhangs, and wherein the duplex has 19 base pairs.

In another embodiment, a siRNA molecule of the invention comprises a single stranded hairpin structure, wherein the siRNA is between about 36 and about 70 (e.g., about 36, 40, 45, 50, 55, 60, 65, or 70) nucleotides in length having between about 18 and about 23 (e.g., about 18, 19, 20, 21, 22, or 23) base pairs, and wherein the siRNA can include a chemical modification comprising a structure having Formula I, Formula II, Formula III and/or Formula IV. For example, an exemplary chemically modified siRNA molecule of the invention comprises a linear oligonucleotide having between about 42 and about 50 (e.g., about 42, 43, 44, 45, 46, 47, 48, 49, or 50) nucleotides that is chemically modified with a chemical modification having Formula I, Formula II, Formula III, and/or Formula IV, wherein the linear oligonucleotide forms a hairpin structure having 19 base pairs and a 2 nucleotide 3′-overhang.

In another embodiment, a linear hairpin siRNA molecule of the invention contains a stem loop motif, wherein the loop portion of the siRNA molecule is biodegradable. For example, a linear hairpin siRNA molecule of the invention is designed such that degradation of the loop portion of the siRNA molecule in vivo can generate a double stranded siRNA molecule with 3′-overhangs, such as 3′-overhangs comprising about 2 nucleotides.

In another embodiment, a siRNA molecule of the invention comprises a circular nucleic acid molecule, wherein the siRNA is between about 38 and about 70 (e.g., about 38, 40, 45, 50, 55, 60, 65, or 70) nucleotides in length having between about 18 and about 23 (e.g., about 18, 19, 20, 21, 22, or 23) base pairs, and wherein the siRNA can include a chemical modification, which comprises a structure having Formula I, Formula II, Formula III and/or Formula IV. For example, an exemplary chemically modified siRNA molecule of the invention comprises a circular oligonucleotide having between about 42 and about 50 (e.g., about 42, 43, 44, 45, 46, 47, 48, 49, or 50) nucleotides that is chemically modified with a chemical modification having Formula I, Formula II, Formula III, and/or Formula IV, wherein the circular oligonucleotide forms a dumbbell shaped structure having 19 base pairs and 2 loops.

In another embodiment, a circular siRNA molecule of the invention contains two loop motifs, wherein one or both loop portions of the siRNA molecule is biodegradable. For example, a circular siRNA molecule of the invention is designed such that degradation of the loop portions of the siRNA molecule in vivo can generate a double stranded siRNA molecule with 3′-overhangs, such as 3′-overhangs comprising about 2 nucleotides.

In one embodiment, a siRNA molecule of the invention comprises at least one (e.g., about 1, 2, 3, 4, 5, 6, 7, 8, 9, 10, or more) abasic residue, for example a compound having Formula V:

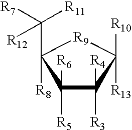

wherein each R3, R4, R5, R6, R7, R8, R10, R11, R12, and R13 is independently H, OH, a substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2.

In one embodiment, a siRNA molecule of the invention comprises at least one (e.g., about 1, 2, 3, 4, 5, 6, 7, 8, 9, 10, or more) inverted abasic residue, for example a compound having Formula VI:

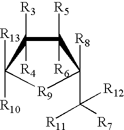

wherein each R3, R4, R5, R6, R7, R8, R10, R11, R12, and R13 is independently H, OH, a substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and either R2, R3, R8 or R13 serve as points of attachment to the siRNA molecule of the invention.

In another embodiment, a siRNA molecule of the invention comprises an abasic residue having Formula II or III, wherein the abasic residue having Formula II or III is connected to the siRNA construct in a 3′,3′, 3′-2′, 2′-3′, or 5′,5′ configuration, such as at the 3′-end, or both 3′ and 5′ ends of one or both siRNA strands.

In one embodiment, a siRNA molecule of the invention comprises one or more (e.g., about 1, 2, 3, 4, 5, 6, 7, 8, 9, 10, or more) locked nucleic acid (LNA) nucleotides, for example at the 5′-end, 3′-end, 5′ and 3′-end, or any combination thereof, of the siRNA molecule.

In another embodiment, a siRNA molecule of the invention comprises one or more (e.g., about 1, 2, 3, 4, 5, 6, 7, 8, 9, 10, or more) acyclic nucleotides, for example at the 5′-end, 3′-end, 5′ and 3′-end, or any combination thereof, of the siRNA molecule.

In one embodiment, the invention features a chemically modified short interfering RNA (siRNA) molecule capable of mediating RNA interference (RNAi) against PTP-TB inside a cell, wherein the chemical modification comprises a conjugate covalently attached to the siRNA molecule. In another embodiment, the conjugate is covalently attached to the siRNA molecule via a biodegradable linker. In one embodiment, the conjugate molecule is attached at the 3′-end of either the sense strand, antisense strand, or both strands of the siRNA. In another embodiment, the conjugate molecule is attached at the 5′-end of either the sense strand, antisense strand, or both strands of the siRNA. In yet another embodiment, the conjugate molecule is attached both the 3′-end and 5′-end of either the sense strand, antisense strand, or both strands of the siRNA, or any combination thereof. In one embodiment, a conjugate molecule of the invention comprises a molecule that facilitates delivery of a siRNA molecule into a biological system such as a cell. In another embodiment, the conjugate molecule attached to the siRNA is a poly ethylene glycol, human serum albumin, or a ligand for a cellular receptor that can mediate cellular uptake. Examples of specific conjugate molecules contemplated by the instant invention that can be attached to siRNA molecules are described in Vargeese et al., U.S. Serial No. 60/311,865, incorporated by reference herein.

In one embodiment, the invention features a siRNA molecule capable of mediating RNA interference (RNAi) against PTP-1B inside a cell, wherein one or both strands of the siRNA comprise ribonucleotides at positions within the siRNA that are critical for siRNA mediated RNAi in a cell. All other positions within the siRNA can include chemically modified nucleotides and/or non-nucleotides such as nucleotides and or non-nucleotides having Formula I, II, III, IV, V, or VI, or any combination thereof to the extent that the ability of the siRNA molecule to support RNAi activity in a cell is maintained.

In one embodiment, the invention features a method for modulating the expression of a PTP-1B gene within a cell, comprising: (a) synthesizing a siRNA molecule of the invention, which can be chemically modified, wherein one of the siRNA strands includes a sequence complementary to RNA of the PTP-1B gene; and (b) introducing the siRNA molecule into a cell under conditions suitable to modulate the expression of the PTP-1B gene in the cell.

In one embodiment, the invention features a method for modulating the expression of a PTP-1B gene within a cell, comprising: (a) synthesizing a siRNA molecule of the invention, which can be chemically modified, wherein one of the siRNA strands includes a sequence complementary to RNA of the PTP-1B gene and wherein the sense strand sequence of the siRNA is identical to the complementary sequence of the PTP-1B RNA; and (b) introducing the siRNA molecule into a cell under conditions suitable to modulate the expression of the PTP-1B gene in the cell.

In another embodiment, the invention features a method for modulating the expression of more than one PTP-1B gene within a cell, comprising: (a) synthesizing siRNA molecules of the invention, which can be chemically modified, wherein one of the siRNA strands includes a sequence complementary to RNA of the PTP-1B genes; and (b) introducing the siRNA molecules into a cell under conditions suitable to modulate the expression of the PTP-1B genes in the cell.

In another embodiment, the invention features a method for modulating the expression of more than one PTP-1B gene within a cell, comprising: (a) synthesizing a siRNA molecule of the invention, which can be chemically modified, wherein one of the siRNA strands includes a sequence complementary to RNA of the PTP-1B gene and wherein the sense strand sequence of the siRNA is identical to the complementary sequence of the PTP-1B RNA; and (b) introducing the siRNA molecules into a cell under conditions suitable to modulate the expression of the PTP-1B genes in the cell.

In one embodiment, the invention features a method of modulating the expression of a PTP-1B gene in a tissue explant, comprising: (a) synthesizing a siRNA molecule of the invention, which can be chemically modified, wherein one of the siRNA strands includes a sequence complementary to RNA of the PTP-1B gene; (b) introducing the siRNA molecule into a cell of the tissue explant derived from a particular organism under conditions suitable to modulate the expression of the PTP-1B gene in the tissue explant, and (c) optionally introducing the tissue explant back into the organism the tissue was derived from or into another organism under conditions suitable to modulate the expression of the PTP-1B gene in that organism.

In one embodiment, the invention features a method of modulating the expression of a PTP-1B gene in a tissue explant, comprising: (a) synthesizing a siRNA molecule of the invention, which can be chemically modified, wherein one of the siRNA strands includes a sequence complementary to RNA of the PTP-1B gene and wherein the sense strand sequence of the siRNA is identical to the complementary sequence of the PTP- 1 B RNA; (b) introducing the siRNA molecule into a cell of the tissue explant derived from a particular organism under conditions suitable to modulate the expression of the PTP-1B gene in the tissue explant, and (c) optionally introducing the tissue explant back into the organism the tissue was derived from or into another organism under conditions suitable to modulate the expression of the PTP-1B gene in that organism.

In another embodiment, the invention features a method of modulating the expression of more than one PTP-1B gene in a tissue explant, comprising: (a) synthesizing siRNA molecules of the invention, which can be chemically modified, wherein one of the siRNA strands includes a sequence complementary to RNA of the PTP-1B genes; (b) introducing the siRNA molecules into a cell of the tissue explant derived from a particular organism under conditions suitable to modulate the expression of the PTP-1B genes in the tissue explant, and (c) optionally introducing the tissue explant back into the organism the tissue was derived from or into another organism under conditions suitable to modulate the expression of the PTP-1B genes in that organism.

In one embodiment, the invention features a method of modulating the expression of a PTP-1B gene in an organism, comprising: (a) synthesizing a siRNA molecule of the invention, which can be chemically modified, wherein one of the siRNA strands includes a sequence complementary to RNA of the PTP-1B gene; and (b) introducing the siRNA molecule into the organism under conditions suitable to modulate the expression of the PTP-1B gene in the organism.

In another embodiment, the invention features a method of modulating the expression of more than one PTP-1B gene in an organism, comprising: (a) synthesizing siRNA molecules of the invention, which can be chemically modified, wherein one of the siRNA strands includes a sequence complementary to RNA of the PTP-1B genes; and (b) introducing the siRNA molecules into the organism under conditions suitable to modulate the expression of the PTP-1B genes in the organism.

The siRNA molecules of the invention can be designed to inhibit PTP-1B gene expression through RNAi targeting of a variety of RNA molecules. In one embodiment, the siRNA molecules of the invention are used to target various RNAs corresponding to a target gene. Non-limiting examples of such RNAs include messenger RNA (mRNA), alternate RNA splice variants of target gene(s), post-transcriptionally modified RNA of target gene(s), pre-mRNA of target gene(s), and/or RNA templates used for PTP-1B activity. If alternate splicing produces a family of transcripts that are distinguished by usage of appropriate exons, the instant invention can be used to inhibit gene expression through the appropriate exons to specifically inhibit or to distinguish among the functions of gene family members. For example, a protein that contains an alternatively spliced transmembrane domain can be expressed in both membrane bound and secreted forms. Use of the invention to target the exon containing the transmembrane domain can be used to determine the functional consequences of pharmaceutical targeting of membrane bound as opposed to the secreted form of the protein. Non-limiting examples of applications of the invention relating to targeting these RNA molecules include therapeutic pharmaceutical applications, pharmaceutical discovery applications, molecular diagnostic and gene function applications, and gene mapping, for example using single nucleotide polymorphism mapping with siRNA molecules of the invention. Such applications can be implemented using known gene sequences or from partial sequences available from an expressed sequence tag (EST).

In another embodiment, the siRNA molecules of the invention are used to target conserved sequences corresponding to a gene family or gene families such as PTP-1B genes. As such, siRNA molecules targeting multiple PTP-1B targets can provide increased therapeutic effect. In addition, siRNA can be used to characterize pathways of gene function in a variety of applications. For example, the present invention can be used to inhibit the activity of target gene(s) in a pathway to determine the function of uncharacterized gene(s) in gene function analysis, mRNA function analysis, or translational analysis. The invention can be used to determine potential target gene pathways involved in various diseases and conditions toward pharmaceutical development. The invention can be used to understand pathways of gene expression involved in development, such as prenatal development, postnatal development and/or aging.

In one embodiment, siRNA molecule(s) and/or methods of the invention are used to inhibit the expression of gene(s) that encode RNA referred to by Genbank Accession number, for example genes such as Genbank Accession No. NM_{—}002827 (PTP-1B). Such sequences are readily obtained using these Genbank Accession numbers.

In one embodiment, the invention features a method comprising: (a) analyzing the sequence of a RNA target encoded by a PTP-TB gene; (b) synthesizing one or more sets of siRNA molecules having sequence complementary to one or more regions of the RNA of (a); and (c) assaying the siRNA molecules of (b) under conditions suitable to determine RNAi targets within the target RNA sequence. In another embodiment, the siRNA molecules of (b) have strands of a fixed length, for example about 23 nucleotides in length. In yet another embodiment, the siRNA molecules of (b) are of differing length, for example having strands of about 19 to about 25 (e.g., about 19, 20, 21, 22, 23, 24, or 25) nucleotides in length.

In one embodiment, the invention features a composition comprising a siRNA molecule of the invention, which can be chemically modified, in a pharmaceutically acceptable carrier or diluent. In another embodiment, the invention features a pharmaceutical composition comprising siRNA molecules of the invention, which can be chemically modified, targeting one or more genes in a pharmaceutically acceptable carrier or diluent. In another embodiment, the invention features a method for treating or preventing a disease or condition in a subject, comprising administering to the subject a composition of the invention under conditions suitable for the treatment or prevention of the disease or condition in the subject, alone or in conjunction with one or more other therapeutic compounds. In yet another embodiment, the invention features a method for reducing or preventing tissue rejection in a subject comprising administering to the subject a composition of the invention under conditions suitable for the reduction or prevention of tissue rejection in the subject.

In another embodiment, the invention features a method for validating a PTP-1B gene target, comprising: (a) synthesizing a siRNA molecule of the invention, which can be chemically modified, wherein one of the siRNA strands includes a sequence complementary to RNA of a PTP-1B target gene; (b) introducing the siRNA molecule into a cell, tissue, or organism under conditions suitable for modulating expression of the PTP-1B target gene in the cell, tissue, or organism; and (c) determining the function of the gene by assaying for any phenotypic change in the cell, tissue, or organism.

In one embodiment, the invention features a kit containing a siRNA molecule of the invention, which can be chemically modified, that can be used to modulate the expression of a PTP-1B target gene in a cell, tissue, or organism. In another embodiment, the invention features a kit containing more than one siRNA molecule of the invention, which can be chemically modified, that can be used to modulate the expression of more than one PTP-1B target gene in a cell, tissue, or organism.

In one embodiment, the invention features a cell containing one or more siRNA molecules of the invention, which can be chemically modified. In another embodiment, the cell containing a siRNA molecule of the invention is a mammalian cell. In yet another embodiment, the cell containing a siRNA molecule of the invention is a human cell.

In one embodiment, the synthesis of a siRNA molecule of the invention, which can be chemically modified, comprises: (a) synthesis of two complementary strands of the siRNA molecule; (b) annealing the two complementary strands together under conditions suitable to obtain a double stranded siRNA molecule. In another embodiment, synthesis of the two complementary strands of the siRNA molecule is by solid phase oligonucleotide synthesis. In yet another embodiment, synthesis of the two complementary strands of the siRNA molecule is by solid phase tandem oligonucleotide synthesis.

In one embodiment, the invention features a method for synthesizing a siRNA duplex molecule comprising: (a) synthesizing a first oligonucleotide sequence strand of the siRNA molecule, wherein the first oligonucleotide sequence strand comprises a cleavable linker molecule that can be used as a scaffold for the synthesis of the second oligonucleotide sequence strand of the siRNA; (b) synthesizing the second oligonucleotide sequence strand of siRNA on the scaffold of the first oligonucleotide sequence strand, wherein the second oligonucleotide sequence strand further comprises a chemical moiety than can be used to purify the siRNA duplex; (c) cleaving the linker molecule of (a) under conditions suitable for the two siRNA oligonucleotide strands to hybridize and form a stable duplex; and (d) purifying the siRNA duplex utilizing the chemical moiety of the second oligonucleotide sequence strand. In another embodiment, cleavage of the linker molecule in (c) above takes place during deprotection of the oligonucleotide, for example under hydrolysis conditions using an alkylamine base such as methylamine. In another embodiment, the method of synthesis comprises solid phase synthesis on a solid support such as controlled pore glass (CPG) or polystyrene, wherein the first sequence of (a) is synthesized on a cleavable linker, such as a succinyl linker, using the solid support as a scaffold. The cleavable linker in (a) used as a scaffold for synthesizing the second strand can comprise similar reactivity as the solid support derivatized linker, such that cleavage of the solid support derivatized linker and the cleavable linker of (a) takes place concomitantly. In another embodiment, the chemical moiety of (b) that can used to isolate the attached oligonucleotide sequence comprises a trityl group, for example a dimethoxytrityl group, which can be employed in a trityl-on synthesis strategy as described herein. In yet another embodiment, the chemical moiety, such as a dimethoxytrityl group, is removed during purification, for example using acidic conditions.

In a further embodiment, the method for siRNA synthesis is a solution phase synthesis or hybrid phase synthesis wherein both strands of the siRNA duplex are synthesized in tandem using a cleavable linker attached to the first sequence which acts a scaffold for synthesis of the second sequence. Cleavage of the linker under conditions suitable for hybridization of the separate siRNA sequence strands results in formation of the double stranded siRNA molecule.

In another embodiment, the invention features a method for synthesizing a siRNA duplex molecule comprising: (a) synthesizing one oligonucleotide sequence strand of the siRNA molecule, wherein the sequence comprises a cleavable linker molecule that can be used as a scaffold for the synthesis of another oligonucleotide sequence; (b) synthesizing a second oligonucleotide sequence having complementarity to the first sequence strand on the scaffold of (a), wherein the second sequence comprises the other strand of the double stranded siRNA molecule and wherein the second sequence further comprises a chemical moiety than can be used to isolate the attached oligonucleotide sequence; (c) purifying the product of (b) utilizing the chemical moiety of the second oligonucleotide sequence strand under conditions suitable for isolating the full length sequence comprising both siRNA oligonucleotide strands connected by the cleavable linker; and (d) under conditions suitable for the two siRNA oligonucleotide strands to hybridize and form a stable duplex. In another embodiment, cleavage of the linker molecule in (c) above takes place during deprotection of the oligonucleotide, for example under hydrolysis conditions. In another embodiment, cleavage of the linker molecule in (c) above takes place after deprotection of the oligonucleotide. In another embodiment, the method of synthesis comprises solid phase synthesis on a solid support such as controlled pore glass (CPG) or polystyrene, wherein the first sequence of (a) is synthesized on a cleavable linker, such as a succinyl linker, using the solid support as a scaffold. The cleavable linker in (a) used as a scaffold for synthesizing the second strand can comprise similar reactivity or differing reactivity as the solid support derivatized linker, such that cleavage of the solid support derivatized linker and the cleavable linker of (a) takes place either concomitantly or sequentially. In another embodiment, the chemical moiety of (b) that can used to isolate the attached oligonucleotide sequence comprises a trityl group, for example a dimethoxytrityl group.

In another embodiment, the invention features a method for making a double stranded siRNA molecule in a single synthetic process, comprising: (a) synthesizing an oligonucleotide having a first and a second sequence, wherein the first sequence is complementary to the second sequence, and the first oligonucleotide sequence is linked to the second sequence via a cleavable linker, and wherein a terminal 5′-protecting group, for example a 5′-O-dimethoxytrityl group (5′-O-DMT) remains on the oligonucleotide having the second sequence; (b) deprotecting the oligonucleotide whereby the deprotection results in the cleavage of the linker joining the two oligonucleotide sequences; and (c) purifying the product of (b) under conditions suitable for isolating the double stranded siRNA molecule, for example using a trityl-on synthesis strategy as described herein.

In one embodiment, the invention features siRNA constructs that mediate RNAi against PTP-1B, wherein the siRNA construct comprises one or more chemical modifications, for example one or more chemical modifications having Formula I, II, III, IV, or V, that increases the nuclease resistance of the siRNA construct.

In another embodiment, the invention features a method for generating siRNA molecules with increased nuclease resistance comprising (a) introducing nucleotides having any of Formula I-VI into a siRNA molecule, and (b) assaying the siRNA molecule of step (a) under conditions suitable for isolating siRNA molecules having increased nuclease resistance.

In one embodiment, the invention features siRNA constructs that mediate RNAi against PTP-1B, wherein the siRNA construct comprises one or more chemical modifications described herein that modulates the binding affinity between the sense and antisense strands of the siRNA construct.

In another embodiment, the invention features a method for generating siRNA molecules with increased binding affinity between the sense and antisense strands of the siRNA molecule comprising (a) introducing nucleotides having any of Formula I-VI into a siRNA molecule, and (b) assaying the siRNA molecule of step (a) under conditions suitable for isolating siRNA molecules having increased binding affinity between the sense and antisense strands of the siRNA molecule.

In one embodiment, the invention features siRNA constructs that mediate RNAi against PTP-1B, wherein the siRNA construct comprises one or more chemical modifications described herein that modulates the binding affinity between the antisense strand of the siRNA construct and a complementary target RNA sequence within a cell.

In another embodiment, the invention features a method for generating siRNA molecules with increased binding affinity between the antisense strand of the siRNA molecule and a complementary target RNA sequence, comprising (a) introducing nucleotides having any of Formula I-VI into a siRNA molecule, and (b) assaying the siRNA molecule of step (a) under conditions suitable for isolating siRNA molecules having increased binding affinity between the antisense strand of the siRNA molecule and a complementary target RNA sequence.

In one embodiment, the invention features siRNA constructs that mediate RNAi against PTP-1B, wherein the siRNA construct comprises one or more chemical modifications described herein that modulate the polymerase activity of a cellular polymerase capable of generating additional endogenous siRNA molecules having sequence homology to the chemically modified siRNA construct.

In another embodiment, the invention features a method for generating siRNA molecules capable of mediating increased polymerase activity of a cellular polymerase capable of generating additional endogenous siRNA molecules having sequence homology to the chemically modified siRNA molecule comprising (a) introducing nucleotides having any of Formula I-VI into a siRNA molecule, and (b) assaying the siRNA molecule of step (a) under conditions suitable for isolating siRNA molecules capable of mediating increased polymerase activity of a cellular polymerase capable of generating additional endogenous siRNA molecules having sequence homology to the chemically modified siRNA molecule.

In one embodiment, the invention features chemically modified siRNA constructs that mediate RNAi against PTP-1B in a cell, wherein the chemical modifications do not significantly effect the interaction of siRNA with a target RNA molecule and/or proteins or other factors that are essential for RNAi in a manner that would decrease the efficacy of RNAi mediated by such siRNA constructs.

In another embodiment, the invention features a method for generating siRNA molecules with improved RNAi activity against PTP-1B, comprising (a) introducing nucleotides having any of Formula I-VI into a siRNA molecule, and (b) assaying the siRNA molecule of step (a) under conditions suitable for isolating siRNA molecules having improved RNAi activity.

In yet another embodiment, the invention features a method for generating siRNA molecules with improved RNAi activity against a PTP-1B target RNA, comprising (a) introducing nucleotides having any of Formula I-VI into a siRNA molecule, and (b) assaying the siRNA molecule of step (a) under conditions suitable for isolating siRNA molecules having improved RNAi activity against the target RNA.

In one embodiment, the invention features siRNA constructs that mediate RNAi against PTP-1B, wherein the siRNA construct comprises one or more chemical modifications described herein that modulates the cellular uptake of the siRNA construct.

In another embodiment, the invention features a method for generating siRNA molecules against PTP-1B with improved cellular uptake, comprising (a) introducing nucleotides having any of Formula I-VI into a siRNA molecule, and (b) assaying the siRNA molecule of step (a) under conditions suitable for isolating siRNA molecules having improved cellular uptake.

In one embodiment, the invention features siRNA constructs that mediate RNAi against PTP-1B, wherein the siRNA construct comprises one or more chemical modifications described herein that increases the bioavailability of the siRNA construct, for example by attaching polymeric conjugates such as polyethyleneglycol or equivalent conjugates that improve the pharmacokinetics of the siRNA construct, or by attaching conjugates that target specific tissue types or cell types in vivo. Non-limiting examples of such conjugates are described in Vargeese et al., U.S. Serial No. 60/311,865 incorporated by reference herein.

In one embodiment, the invention features a method for generating siRNA molecules of the invention with improved bioavailability, comprising (a) introducing a conjugate into the structure of a siRNA molecule, and (b) assaying the siRNA molecule of step (a) under conditions suitable for isolating siRNA molecules having improved bioavailability. Such conjugates can include ligands for cellular receptors such as peptides derived from naturally occurring protein ligands, protein localization sequences including cellular ZIP code sequences, antibodies, nucleic acid aptamers, vitamins and other co-factors such as folate and N-acetylgalactosamine, polymers such as polyethyleneglycol (PEG), phospholipids, polyamines such as spermine or spermidine, and others.

In another embodiment, the invention features a method for generating siRNA molecules of the invention with improved bioavailability, comprising (a) introducing an excipient formulation to a siRNA molecule, and (b) assaying the siRNA molecule of step (a) under conditions suitable for isolating siRNA molecules having improved bioavailability. Such excipients include polymers such as cyclodextrins, lipids, cationic lipids, polyamines, phospholipids, and others.

In another embodiment, the invention features a method for generating siRNA molecules of the invention with improved bioavailability, comprising (a) introducing nucleotides having any of Formula I-VI into a siRNA molecule, and (b) assaying the siRNA molecule of step (a) under conditions suitable for isolating siRNA molecules having improved bioavailability.

In another embodiment, polyethylene glycol (PEG) can be covalently attached to siRNA compounds of the present invention. The attached PEG can be any molecular weight, preferably from about 2,000 to about 50,000 daltons (Da).

The present invention can be used alone or as a component of a kit having at least one of the reagents necessary to carry out the in vitro or in vivo introduction of RNA to test samples and/or subjects. For example, preferred components of the kit include the siRNA and a vehicle that promotes introduction of the siRNA. Such a kit can also include instructions to allow a user of the kit to practice the invention.

The term “short interfering RNA” or “siRNA” as used herein refers to a double stranded nucleic acid molecule capable of RNA interference “RNAi”, see for example Bass, 2001, Nature, 411, 428-429; Elbashir et al., 2001, Nature, 411, 494-498; and Kreutzer et al., International PCT Publication No. WO00/44895; Zernicka-Goetz et al., International PCT Publication No. WO 01/36646; Fire, International PCT Publication No. WO99/32619; Plaetinck et al., International PCT Publication No. WO00/01846; Mello and Fire, International PCT Publication No. WO01/29058; Deschamps-Depaillette, International PCT Publication No. WO99/07409; and Li et al., International PCT Publication No. WO00/44914. As used herein, siRNA molecules need not be limited to those molecules containing only RNA, but further encompasses chemically modified nucleotides and non-nucleotides.

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

By “inhibit” it is meant that the activity of a gene expression product or level of RNAs or equivalent RNAs encoding one or more gene products is reduced below that observed in the absence of the nucleic acid molecule of the invention. In one embodiment, inhibition with a siRNA molecule preferably is below that level observed in the presence of an inactive or attenuated molecule that is unable to mediate an RNAi response. In another embodiment, inhibition of gene expression with the siRNA molecule of the instant invention is greater in the presence of the siRNA molecule than in its absence.

By “gene” or “target gene” is meant, a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates. Non-limiting examples of fungi include molds or yeasts.

By “PTP-1B” as used herein is meant, any protein, peptide, or polypeptide, having protein tyrosine phosphatase-1B activity, such as phosphorylation of insulin receptors.

By “highly conserved sequence region” is meant, a nucleotide sequence of one or more regions in a target gene does not vary significantly from one generation to the other or from one biological system to the other.

By “complementarity” or “complementary” is meant that a nucleic acid can form hydrogen bond(s) with another nucleic acid sequence by either traditional Watson-Crick or other non-traditional types of interaction. In reference to the nucleic molecules of the present invention, the binding free energy for a nucleic acid molecule with its complementary sequence is sufficient to allow the relevant function of the nucleic acid to proceed, e.g., RNAi activity. For example, the degree of complementarity between the sense and antisense strand of the siRNA construct can be the same or different from the degree of complementarity between the antisense strand of the siRNA and the target RNA sequence. Complementarity to the target sequence of less than 100% in the antisense strand of the siRNA duplex, including point mutations, is reported not to be tolerated when these changes are located between the 3′-end and the middle of the antisense siRNA (completely abolishes siRNA activity), whereas mutations near the 5′-end of the antisense siRNA strand can exhibit a small degree of RNAi activity (Elbashir et al., 2001, The EMBO Journal, 20, 6877-6888). Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The siRNA molecules of the invention represent a novel therapeutic approach to treat a variety of pathologic indications, including Type I diabetes, Type II diabetes, obesity and/or any other diseases or conditions that are related to the levels of PTP-1B in a cell or tissue, alone or in combination with other therapies. The reduction of PTP-1B expression (specifically PTP-1B RNA levels) and thus reduction in the level of the respective protein relieves, to some extent, the symptoms of the disease or condition.

In one embodiment of the present invention, each sequence of a siRNA molecule of the invention is independently about 18 to about 24 nucleotides in length, in specific embodiments about 18, 19, 20, 21, 22, 23, or 24 nucleotides in length. In another embodiment, the siRNA duplexes of the invention independently comprise between about 17 and about 23 (e.g., about 17, 18, 19, 20, 21, 22, or 23) base pairs. In yet another embodiment, siRNA molecules of the invention comprising hairpin or circular structures are about 35 to about 55 (e.g., about 35, 40, 45, 50, or 55) nucleotides in length, or about 38 to about 44 (e.g., about 38, 39, 40, 41, 42, 43, or 44) nucleotides in length and comprising about 16 to about 22 (e.g., about 16, 17, 18, 19, 20, 21, or 22) base pairs. Exemplary siRNA molecules of the invention are shown in Table I (all sequences are shown 5′-3′) and/or FIGS. 4 and 5.

As used herein “cell” is used in its usual biological sense, and does not refer to an entire multicellular organism, e.g., specifically does not refer to a human. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell.

The siRNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Table I and/or FIGS. 4 and 5. Examples of such nucleic acid molecules consist essentially of sequences defined in this table.

In another aspect, the invention provides mammalian cells containing one or more siRNA molecules of this invention. The one or more siRNA molecules can independently be targeted to the same or different sites.

By “RNA” is meant a molecule comprising at least one ribonucleotide residue. By “ribonucleotide” is meant a nucleotide with a hydroxyl group at the 2′ position of a β-D-ribo-furanose moiety. The terms include double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

The term “phosphorothioate” as used herein refers to an internucleotide linkage having Formula I, wherein Z and/or W comprise a sulfur atom. Hence, the term phosphorothioate refers to both phosphorothioate and phosphorodithioate internucleotide linkages.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein refers to any nucleotide having an acyclic ribose sugar, for example where any of the ribose carbons (C1, C2, C3, C4, or C5), are independently or in combination absent from the nucleotide.

The nucleic acid molecules of the instant invention, individually, or in combination or in conjunction with other drugs, can be used to treat diseases or conditions discussed herein. For example, to treat a particular disease or condition, the siRNA molecules can be administered to a subject or can be administered to other appropriate cells evident to those skilled in the art, individually or in combination with one or more drugs under conditions suitable for the treatment.

In a further embodiment, the siRNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siRNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

In one embodiment, the invention features an expression vector comprising a nucleic acid sequence encoding at least one siRNA molecule of the invention, in a manner which allows expression of the siRNA molecule. For example, the vector can contain sequence(s) encoding both strands of a siRNA molecule comprising a duplex. The vector can also contain sequence(s) encoding a single nucleic acid molecule that is self complementary and thus forms a siRNA molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725.

In another embodiment, the invention features a mammalian cell, for example, a human cell, including an expression vector of the invention.

In yet another embodiment, the expression vector of the invention comprises a sequence for a siRNA molecule having complementarity to a RNA molecule referred to by a Genbank Accession numbers, for example genes such as Genbank Accession No. NM_{—}002827 (PTP-1B).

In one embodiment, an expression vector of the invention comprises a nucleic acid sequence encoding two or more siRNA molecules, which can be the same or different.

In another aspect of the invention, siRNA molecules that interact with target RNA molecules and down-regulate gene encoding target RNA molecules (for example target RNA molecules referred to by Genbank Accession numbers herein) are expressed from transcription units inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. The recombinant vectors capable of expressing the siRNA molecules can be delivered as described herein, and persist in target cells. Alternatively, viral vectors can be used that provide for transient expression of siRNA molecules. Such vectors can be repeatedly administered as necessary. Once expressed, the siRNA molecules bind and down-regulate gene function or expression via RNA interference (RNAi). Delivery of siRNA expressing vectors can be systemic, such as by intravenous or intramuscular administration, by administration to target cells ex-planted from a subject followed by reintroduction into the subject, or by any other means that would allow for introduction into the desired target cell.

By “vectors” is meant any nucleic acid- and/or viral-based technique used to deliver a desired nucleic acid.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows a non-limiting example of a scheme for the synthesis of siRNA molecules. The complementary siRNA sequence strands, strand 1 and strand 2, are synthesized in tandem and are connected by a cleavable linkage, such as a nucleotide succinate or abasic succinate, which can be the same or different from the cleavable linker used for solid phase synthesis on a solid support. The synthesis can be either solid phase or solution phase, in the example shown, the synthesis is a solid phase synthesis. The synthesis is performed such that a protecting group, such as a dimethoxytrityl group, remains intact on the terminal nucleotide of the tandem oligonucleotide. Upon cleavage and deprotection of the oligonucleotide, the two siRNA strands spontaneously hybridize to form a siRNA duplex, which allows the purification of the duplex by utilizing the properties of the terminal protecting group, for example by applying a trityl on purification method wherein only duplexes/oligonucleotides with the terminal protecting group are isolated.

FIG. 2 shows a MALDI-TOV mass spectrum of a purified siRNA duplex synthesized by a method of the invention. The two peaks shown correspond to the predicted mass of the separate siRNA sequence strands. This result demonstrates that the siRNA duplex generated from tandem synthesis can be purified as a single entity using a simple trityl-on purification methodology.

FIG. 3 shows a non-limiting proposed mechanistic representation of target RNA degradation involved in RNAi. Double stranded RNA (dsRNA), which is generated by RNA dependent RNA polymerase (RdRP) from foreign single stranded RNA, for example viral, transposon, or other exogenous RNA, activates the DICER enzyme which in turn generates siRNA duplexes having terminal phosphate groups (P). An active siRNA complex forms which recognizes a target RNA, resulting in degradation of the target RNA by the RISC endonuclease complex or in the synthesis of additional RNA by RNA dependent RNA polymerase (RdRP), which can activate DICER and result in additional siRNA molecules, thereby amplifying the RNAi response.

FIG. 4 shows non-limiting examples of chemically modified siRNA constructs of the present invention. In the figure, N stands for any nucleotide (adenosine, guanosine, cytosine, uridine, or optionally thymidine, for example thymidine can be substituted in the overhanging regions designated by parenthesis (N N). Various modifications are shown for the sense and antisense strands of the siRNA constructs. A The sense strand comprises 21 nucleotides having four phosphorothioate 5′ and 3′-terminal internucleotide linkages, wherein the two terminal 3′-nucleotides are optionally base paired and wherein all pyrimidine nucleotides that may be present are 2′-O-methyl modified nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complimentary to the target RNA sequence, and having one 3′-terminal phosphorothioate internucleotide linkage and four 5′-terminal phosphorothioate internucleotide linkages and wherein all pyrimidine nucleotides that may be present are 2′-deoxy-2′-fluoro modified nucleotides except for (N N) nucleotides, which can comprise naturally occurring ribonucleotides, deoxynucleotides, universal bases, or other chemical modifications described herein. B The sense strand comprises 21 nucleotides wherein the two terminal 3′-nucleotides are optionally base paired and wherein all pyrimidine nucleotides that may be present are 2′-O-methyl modified nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complimentary to the target RNA sequence, and wherein all pyrimidine nucleotides that may be present are 2′-deoxy-2′-fluoro modified nucleotides except for (N N) nucleotides, which can comprise naturally occurring ribonucleotides, deoxynucleotides, universal bases, or other chemical modifications described herein. C The sense strand comprises 21 nucleotides having 5′- and 3′-terminal cap moieties wherein the two terminal 3′-nucleotides are optionally base paired and wherein all pyrimidine nucleotides that may be present are 2′-O-methyl modified nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complimentary to the target RNA sequence, and wherein all pyrimidine nucleotides that may be present are 2′-deoxy-2′-fluoro modified nucleotides except for (N N) nucleotides, which can comprise naturally occurring ribonucleotides, deoxynucleotides, universal bases, or other chemical modifications described herein. D The sense strand comprises 21 nucleotides having five phosphorothioate 5′ and 3′-terminal internucleotide linkages, wherein the two terminal 3′-nucleotides are optionally base paired and wherein all nucleotides are ribonucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complimentary to the target RNA sequence, and having one 3′-terminal phosphorothioate internucleotide linkage and five 5′-terminal phosphorothioate internucleotide linkages and wherein all nucleotides are ribonucleotides except for (N N) nucleotides, which can comprise naturally occurring ribonucleotides, deoxynucleotides, universal bases, or other chemical modifications described herein. E The sense strand comprises 21 nucleotides wherein the two terminal 3′-nucleotides are optionally base paired and wherein all pyrimidine nucleotides that may be present are 2′-O-methyl nucleotides except for (N N) nucleotides, which can comprise naturally occurring ribonucleotides, deoxynucleotides, universal bases, or other chemical modifications described herein. The antisense strand comprises 21 nucleotides all having phosphorothioate internucleotide linkages, wherein the two terminal 3′-nucleotides are optionally complimentary to the target RNA sequence, and wherein all nucleotides are ribonucleotides except for (N N) nucleotides, which can comprise naturally occurring ribonucleotides, deoxynucleotides, universal bases, or other chemical modifications described herein. F The sense strand comprises 21 nucleotides having 5′- and 3′-terminal cap moieties, wherein the two terminal 3′-nucleotides are optionally base paired and wherein all pyrimidine nucleotides that may be present are 2′-O-methyl nucleotides except for (N N) nucleotides, which can comprise naturally occurring ribonucleotides, deoxynucleotides, universal bases, or other chemical modifications described herein. The antisense strand comprises 21 nucleotides, wherein the two terminal 3′-nucleotides are optionally complimentary to the target RNA sequence, and having one 3′-terminal phosphorothioate internucleotide linkage and wherein all pyrimidine nucleotides that may be present are 2′-deoxy-2′-fluoro nucleotides except for (N N) nucleotides, which can comprise naturally occurring ribonucleotides, deoxynucleotides, universal bases, or other chemical modifications described herein. The antisense strand of constructs A-F comprise sequence complimentary to target RNA sequence of the invention.

FIG. 5 shows non-limiting examples of specific chemically modified siRNA sequences of the invention. A-F applies the chemical modifications described in FIG. 4A-F to a PTP-1B siRNA sequence.

FIG. 6 shows non-limiting examples of different siRNA constructs of the invention. The examples shown (constructs 1, 2, and 3) have 19 representative base pairs, however, different embodiments of the invention include any number of base pairs described herein. Bracketed regions represent nucleotide overhangs, for example comprising between about 1, 2, 3, or 4 nucleotides in length, preferably about 2 nucleotides. Constructs 1 and 2 can be used independently for RNAi activity. Construct 2 can comprise a polynucleotide or non-nucleotide linker, which can optionally be designed as a biodegradable linker. In one embodiment, the loop structure shown in construct 2 can comprise a biodegradable linker that results in the formation of construct 1 in vivo and/or in vitro. In another example, construct 3 can be used to generate construct 2 under the same principle wherein a linker is used to generate the active siRNA construct 2 in vivo and/or in vitro, which can optionally utilize another biodegradable linker to generate the active siRNA construct 1 in vivo and/or in vitro. As such, the stability and/or activity of the siRNA constructs can be modulated based on the design of the siRNA construct for use in vivo or in vitro and/or in vitro.

### Mechanism of Action of Nucleic Acid Molecules of the Invention

RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

### Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., individual siRNA oligonucleotide sequences or siRNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides or 2′-deoxy-2′-fluoro nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M =6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for RNA including certain siRNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 lL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA-3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siRNA molecules of the invention can also be synthesized via a tandem synthesis methodology as described in Example 1 herein, wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker. The tandem synthesis of siRNA as described herein can be readily adapted to both multiwell/multiplate synthesis platforms such as 96 well or similarly larger multi-well platforms. The tandem synthesis of siRNA as described herein can also be readily adapted to large scale synthesis platforms employing batch reactors, synthesis columns and the like.

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, T1BS 17, 34; Usman et al., 1994, Nucleic Acids Symp. Ser. 31, 163). siRNA constructs can be purified by gel electrophoresis using general methods or can be purified by high pressure liquid chromatography (HPLC; see Wincott et al., supra, the totality of which is hereby incorporated herein by reference) and re-suspended in water.

In another aspect of the invention, siRNA molecules of the invention are expressed from transcription units inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. The recombinant vectors capable of expressing the siRNA molecules can be delivered as described herein, and persist in target cells. Alternatively, viral vectors can be used that provide for transient expression of siRNA molecules.

### Optimizing Activity of the Nucleic Acid Molecule of the Invention

Chemically synthesizing nucleic acid molecules with modifications (base, sugar and/or phosphate) can prevent their degradation by serum ribonucleases, which can increase their potency (see e.g., Eckstein et al., International Publication No. WO92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO93/15187; and Rossi et al., International Publication No. WO91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO93/15187; Sproat, U.S. Pat. No. 5,334,711 and Beigelman et al., 1995, J. Biol. Chem., 270, 25702; Beigelman et al., International PCT publication No. WO97/26270; Beigelman et al., U.S. Pat. No. 5,716,824; Usman et al., U.S. Pat. No. 5,627,053; Woolf et al., International PCT Publication No. WO98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into nucleic acid molecules without modulating catalysis, and are incorporated by reference herein. In view of such teachings, similar modifications can be used as described herein to modify the siRNA nucleic acid molecules of the instant invention so long as the ability of siRNA to promote RNAi is cells is not significantly inhibited.

While chemical modification of oligonucleotide internucleotide linkages with phosphorothioate, phosphorothioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

Small interfering RNA (siRNA) molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability, as described above.

In one embodiment, nucleic acid molecules of the invention include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more (e.g., about 1, 2, 3, 4, 5, 6, 7, 8, 9, 10, or more) LNA “locked nucleic acid” nucleotides such as a 2′, 4′-C mythylene bicyclo nucleotide (see for example Wengel et al., International PCT Publication No. WO00/66604 and WO 99/14226).

In another embodiment, the invention features conjugates and/or complexes of siRNA molecules of the invention. Such conjugates and/or complexes can be used to facilitate delivery of siRNA molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example, 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siRNA molecules either alone or in combination with the molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

Therapeutic nucleic acid molecules (e.g., siRNA molecules) delivered exogenously optimally are stable within cells until reverse trascription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, siRNA molecules having chemical modifications that maintain or enhance enzymatic activity of proteins involved in RNAi are provided. Such nucleic acids are also generally more resistant to nucleases than unmodified nucleic acids. Thus, in vitro and/or in vivo the activity should not be significantly lowered.

Use of the nucleic acid-based molecules of the invention will lead to better treatment of the disease progression by affording the possibility of combination therapies (e.g., multiple siRNA molecules targeted to different genes; nucleic acid molecules coupled with known small molecule modulators; or intermittent treatment with combinations of molecules, including different motifs and/or other chemical or biological molecules). The treatment of subjects with siRNA molecules can also include combinations of different types of nucleic acid molecules, such as enzymatic nucleic acid molecules (ribozymes), allozymes, antisense, 2,5-A oligoadenylate, decoys, aptamers etc.

In another aspect a siRNA molecule of the invention comprises one or more 5′ and/or a 3′- cap structure, for example on only the sense siRNA strand, antisense siRNA strand, or both siRNA strands.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

An “alkyl” group refers to a saturated aliphatic hydrocarbon, including straight-chain, branched-chain, and cyclic alkyl groups. Preferably, the alkyl group has 1 to 12 carbons. More preferably, it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups that are unsaturated hydrocarbon groups containing at least one carbon-carbon double bond, including straight-chain, branched-chain, and cyclic groups. Preferably, the alkenyl group has 1 to 12 carbons. More preferably, it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups that have an unsaturated hydrocarbon group containing at least one carbon-carbon triple bond, including straight-chain, branched-chain, and cyclic groups. Preferably, the alkynyl group has 1 to 12 carbons. More preferably, it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO92/07065; Usman et al., International PCT Publication No. WO93/15187; Uhlman & Peyrnan, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

In one embodiment, the invention features modified siRNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, fornacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH2, which may be modified or unmodified. Such modified groups are described, for example, in Eckstein et al., U.S. 5,672,695 and Matulic-Adamic et al., U.S. Pat. No. 6,248,878, which are both incorporated by reference in their entireties.

Various modifications to nucleic acid siRNA structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

### Administration of Nucleic Acid Molecules

A siRNA molecule of the invention can be adapted for use to treat Alzheimer's disease. For example, a siRNA molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713 and Sullivan et al., PCT WO94/02595, further describes the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other delivery vehicles, such as hydrogels, cyclodextrins, biodegradable nanocapsules, and bioadhesive microspheres, or by proteinaceous vectors (O'Hare and Normand, International PCT Publication No. WO00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO99/31262. Many examples in the art describe CNS delivery methods of oligonucleotides by osmotic pump, (see Chun et al., 1998, Neuroscience Letters, 257, 135-138, D'Aldin et al., 1998, Mol. Brain Research, 55, 151-164, Dryden et al., 1998, J. Endocrinol., 157, 169-175, Ghirnikar et al., 1998, Neuroscience Letters, 247, 21-24) or direct infusion (Broaddus et al., 1997, Neurosurg. Focus, 3, article 4). Other routes of delivery include, but are not limited to oral (tablet or pill form) and/or intrathecal delivery (Gold, 1997, Neuroscience, 76, 1153-1158). More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

Thus, the invention features a pharmaceutical composition comprising one or more nucleic acid(s) of the invention in an acceptable carrier, such as a stabilizer, buffer, and the like. The polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the siRNA molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al., 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO96/10391; Ansell et al., International PCT Publication No. WO96/10390; Holland et al., International PCT Publication No. WO96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxynethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention may also be administered to a subject in combination with other therapeutic compounds to increase the overall therapeutic effect. The use of multiple compounds to treat an indication may increase the beneficial effects while reducing the presence of side effects.

In one embodiment, the invention compositions suitable for administering nucleic acid molecules of the invention to specific cell types, such as hepatocytes. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention.

Alternatively, certain siRNA molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev, 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO93/23569, and Sullivan et al., PCT WO94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

In another aspect of the invention, RNA molecules of the present invention can be expressed from transcription units (see for example Couture et al., 1996, TIG., 12, 510) inserted into DNA or RNA vectors. The recombinant vectors can be DNA plasmids or viral vectors. siRNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886). The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target mRNA and generates an RNAi response. Delivery of siRNA molecule expressing vectors can be systemic, such as by intravenous or intramuscular administration, by administration to target cells ex-planted from a subject followed by reintroduction into the subject, or by any other means that would allow for introduction into the desired target cell (for a review see Couture et al., 1996, TIG., 12, 510).

In one aspect the invention features an expression vector comprising a nucleic acid sequence encoding at least one siRNA molecule of the instant invention. The expression vector can encode one or both strands of a siRNA duplex, or a single self complementary strand that self hybridizes into a siRNA duplex. The nucleic acid sequences encoding the siRNA molecules of the instant invention can be operably linked in a manner that allows expression of the siRNA molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siRNA molecules of the instant invention; wherein said sequence is operably linked to said initiation region and said termination region, in a manner that allows expression and/or delivery of the siRNA molecule. The vector can optionally include an open reading frame (ORF) for a protein operably linked on the 5′ side or the 3′-side of the sequence encoding the siRNA of the invention; and/or an intron (intervening sequences).

Transcription of the siRNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siRNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO96/18736. The above siRNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising a nucleic acid sequence encoding at least one of the siRNA molecules of the invention, in a manner that allows expression of that siRNA molecule. The expression vector comprises in one embodiment; a) a transcription initiation region; b) a transcription termination region; and c) a nucleic acid sequence encoding at least one strand of the siRNA molecule; wherein the sequence is operably linked to the initiation region and the termination region, in a manner that allows expression and/or delivery of the siRNA molecule.

In another embodiment the expression vector comprises: a) a transcription initiation region; b) a transcription termination region; c) an open reading frame; and d) a nucleic acid sequence encoding at least one strand of a siRNA molecule, wherein the sequence is operably linked to the 3′-end of the open reading frame; and wherein the sequence is operably linked to the initiation region, the open reading frame and the termination region, in a manner that allows expression and/or delivery of the siRNA molecule. In yet another embodiment the expression vector comprises: a) a transcription initiation region; b) a transcription termination region; c) an intron; and d) a nucleic acid sequence encoding at least one siRNA molecule; wherein the sequence is operably linked to the initiation region, the intron and the termination region, in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siRNA molecule, wherein the sequence is operably linked to the 3′-end of the open reading frame; and wherein the sequence is operably linked to the initiation region, the intron, the open reading frame and the termination region, in a manner which allows expression and/or delivery of the siRNA molecule.

### EXAMPLES

The following are non-limiting examples showing the selection, isolation, synthesis and activity of nucleic acids of the instant invention.

### Example 1

### Tandem Synthesis of siRNA Constructs

Exemplary siRNA molecules of the invention are synthesized in tandem using a cleavable linker, for example a succinyl-based linker. Tandem synthesis as described herein is followed by a one step purification process that provides RNAi molecules in high yield. This approach is highly amenable to siRNA synthesis in support of high throughput RNAi screening, and can be readily adapted to multi-column or multi-well synthesis platforms.

After completing a tandem synthesis of an siRNA oligo and its compliment in which the 5′-terminal dimethoxytrityl (5′-O-DMT) group remains intact (trityl on synthesis), the oligonucleotides are deprotected as described above. Following deprotection, the siRNA sequence strands are allowed to spontaneouslyhybridize. This hybridization yields a duplex in which one strand has retained the 5′-O-DMT group while the complementary strand comprises a terminal 5′-hydroxyl. The newly formed duplex to behaves as a single molecule during routine solid-phase extraction purification (Trityl-On purification) even though only one molecule has a dimethoxytrityl group. Because the strands form a stable duplex, this dimethoxytrityl group (or an equivalent group, such as other trityl groups or other hydrophobic moieties) is all that is required to purify the pair of oligos, for example by using a C18 cartridge.

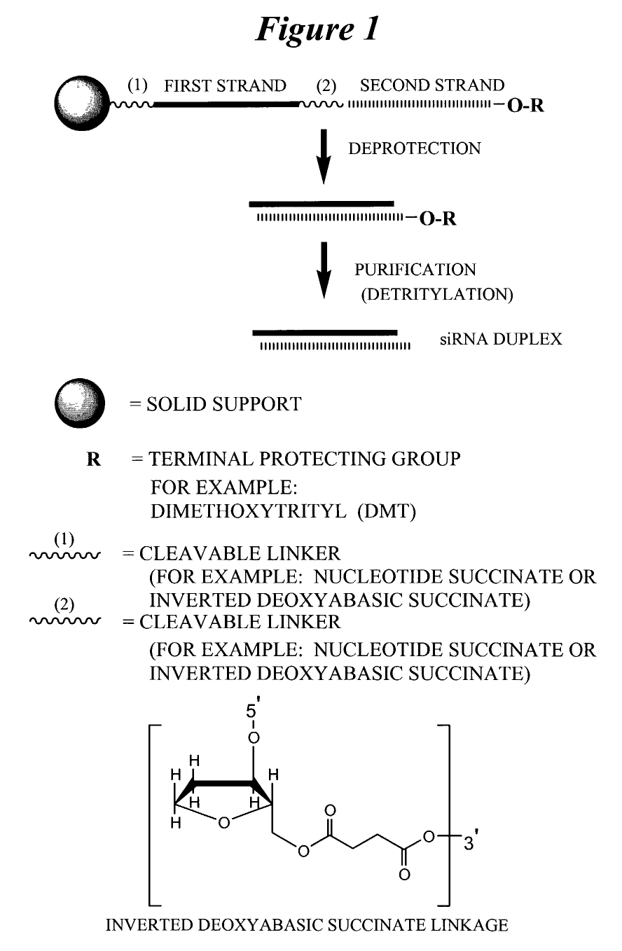

Standard phosphoramidite synthesis chemistry is used up to point of introducing a tandem linker, such as an inverted deoxyabasic succinate linker (see FIG. 1) or an equivalent cleavable linker. A non-limiting example of linker coupling conditions that can be used includes a hindered base such as diisopropylethylamine (DIPA) and/or DMAP in the presence of an activator reagent such as Bromotripyrrolidinophosphoniumhexaflurorophosphate (PyBrOP). After the linker is coupled, standard synthesis chemistry is utilized to complete synthesis of the second sequence leaving the terminal the 5′-O-DMT intact. Following synthesis, the resulting oligonucleotide is deprotected according to the procedures described herein and quenched with a suitable buffer, for example with 50 mM NaOAc or 1.5 M NH₄H₂CO₃.

Purification of the siRNA duplex can be readily accomplished using solid phase extraction, for example using a Waters C18 SepPak 1 g cartridge conditioned with 1 column volume (CV) of acetonitrile, 2 CV H2O, and 2 CV 50 mM NaOAc. The sample is loaded and then washed with 1 CV H2O or 50 mM NaOAc. Failure sequences are eluted with 1 CV 14% ACN (Aqueous with 50 mM NaOAc and 50 mM NaCl). The column is then washed, for example with 1 CV H2O followed by on-column detritylation, for example by passing 1 CV of 1% aqueous trifluoroacetic acid (TFA) over the column, then adding a second CV of 1% aqueous TFA to the column and allowing to stand for approx. 10 minutes. The remaining TFA solution is removed and the column washed with H2O followed by 1 CV IM NaCl and additional H2O. The siRNA duplex product is then eluted, for example using 1 CV 20% aqueous CAN.

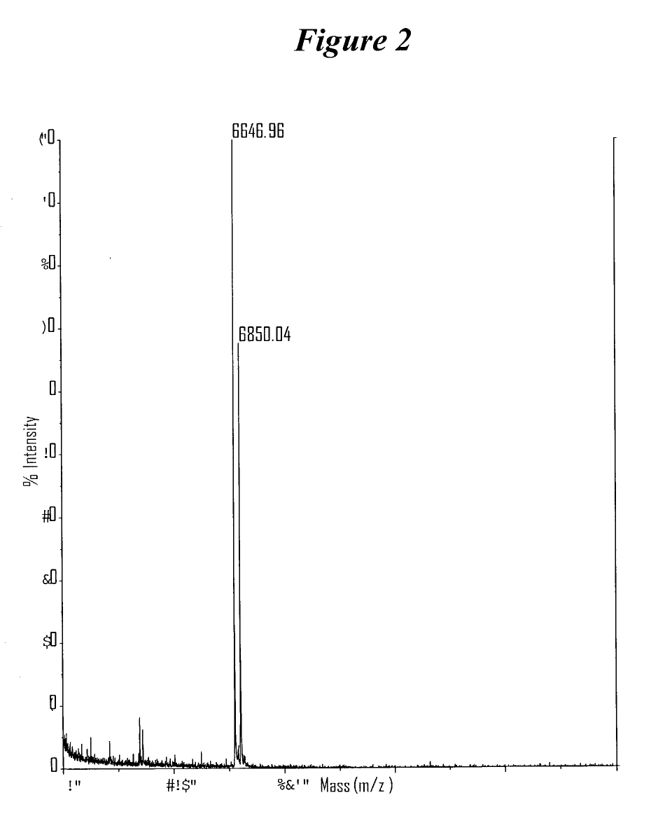

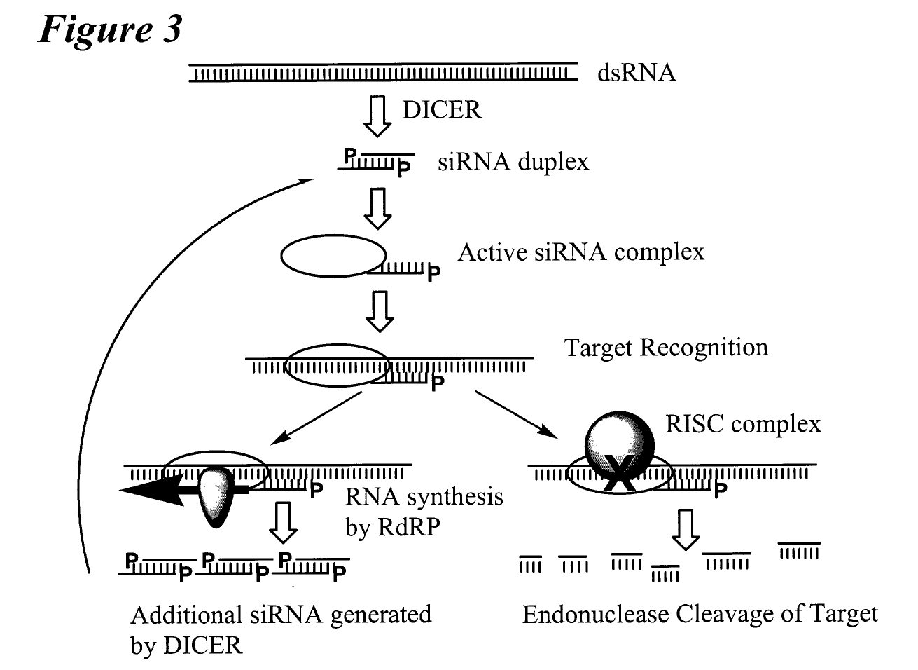

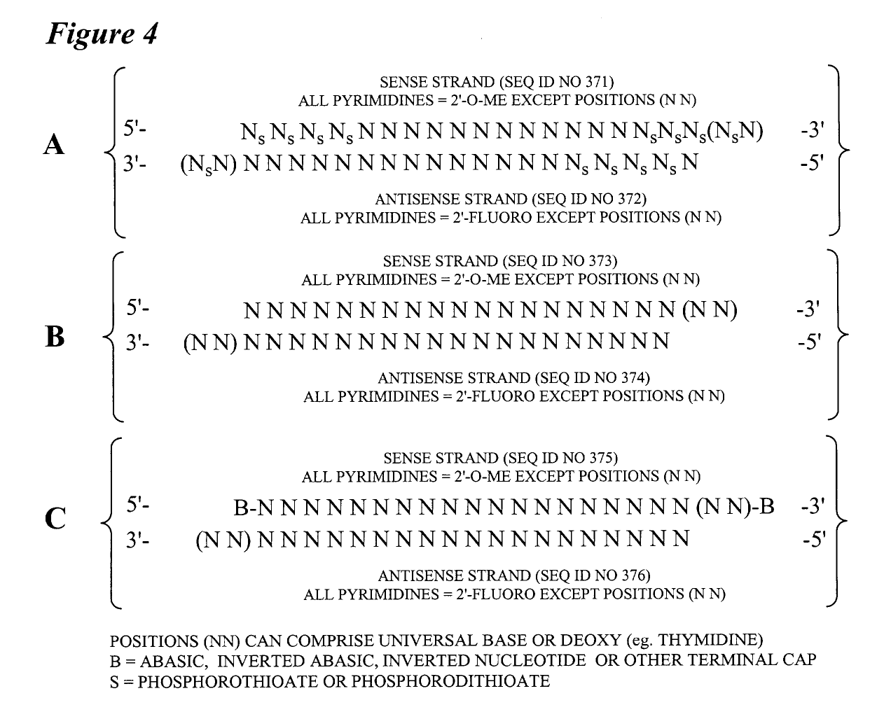

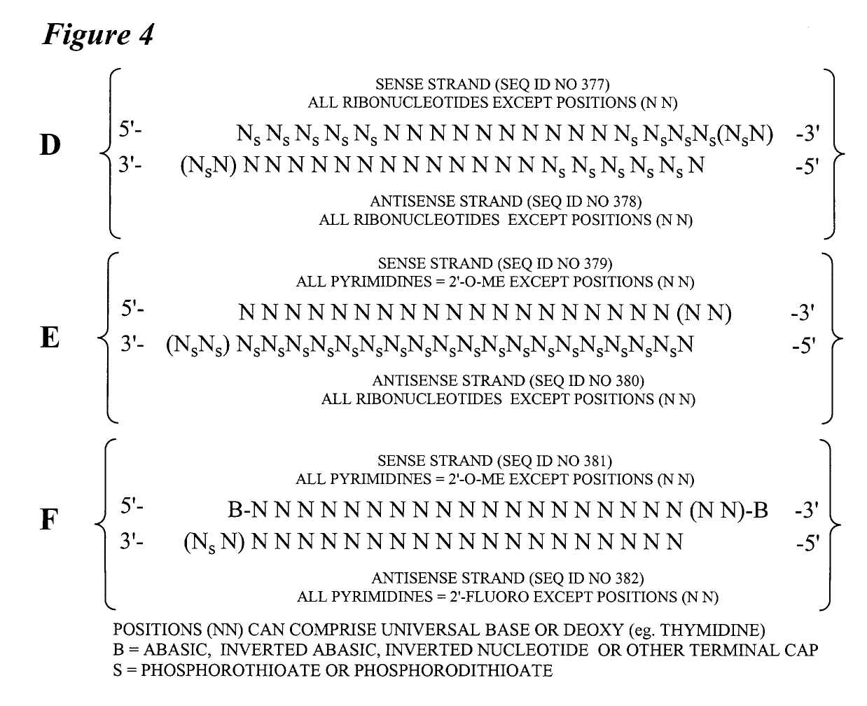

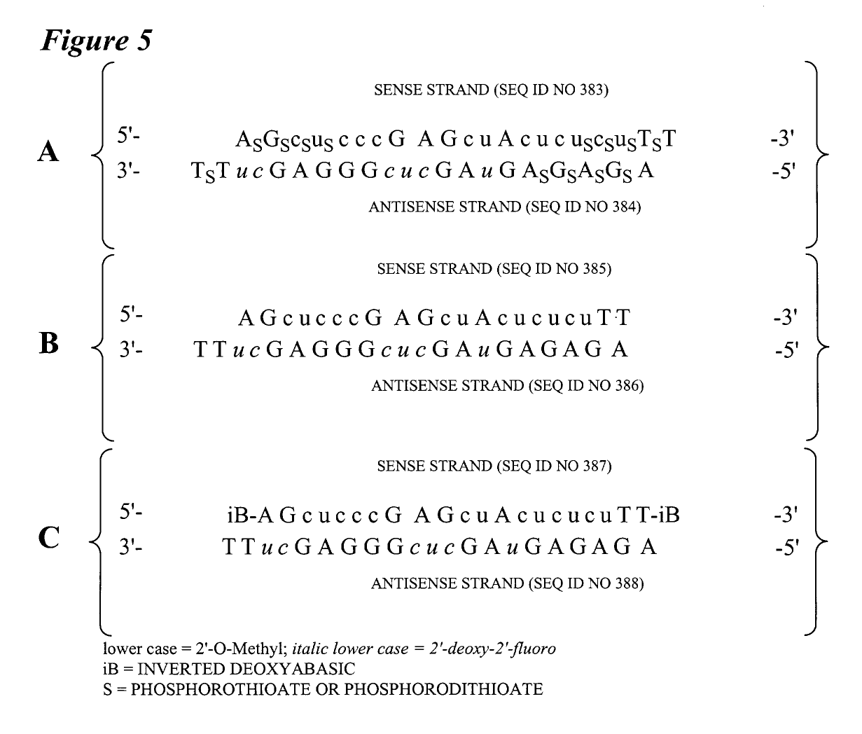

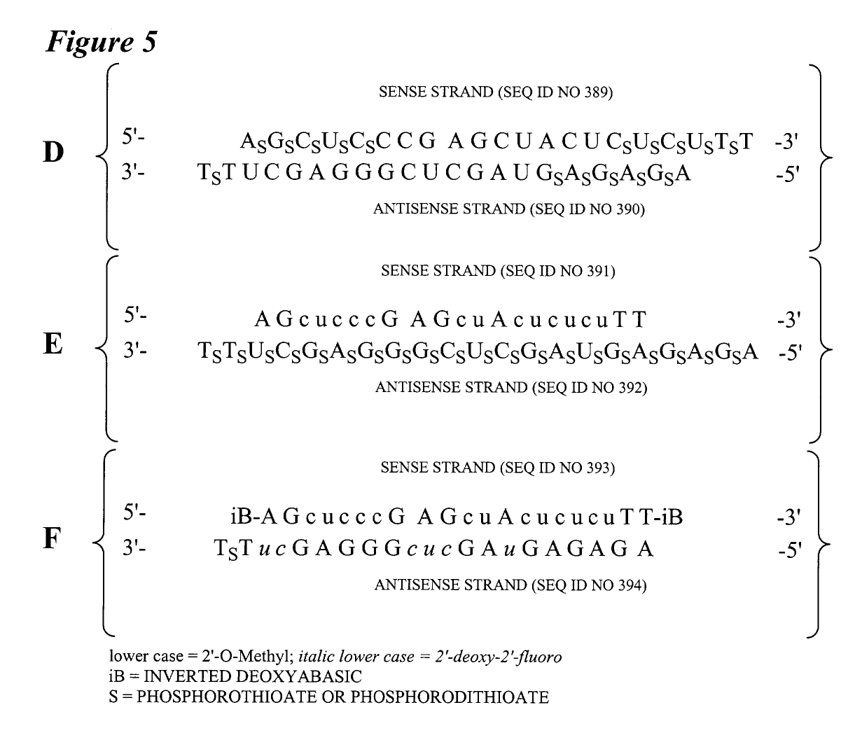

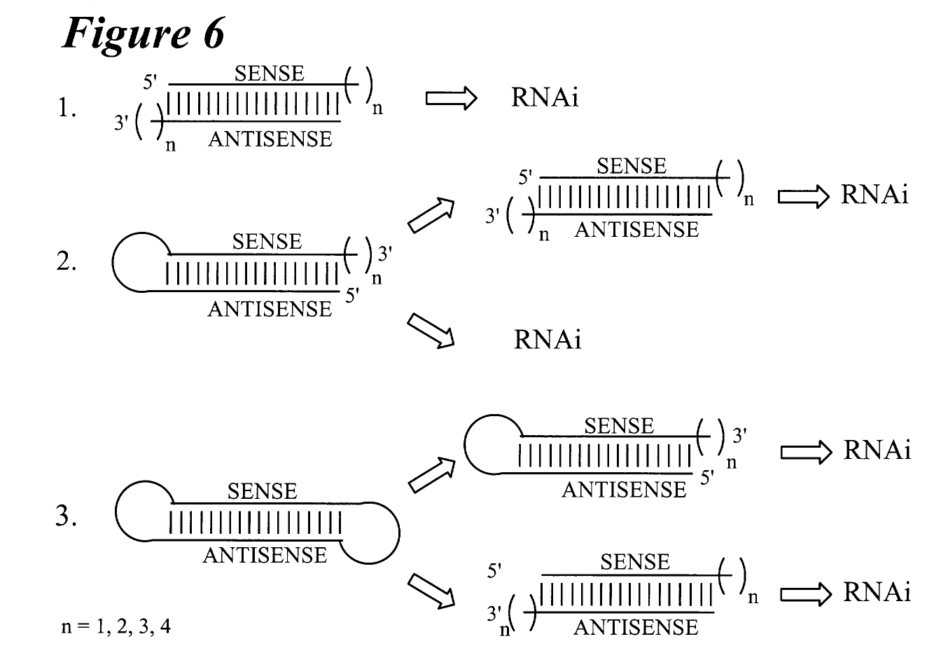

FIG. 2 provides an example of MALDI-TOV mass spectrometry analysis of a purified siRNA construct in which each peak corresponds to the calculated mass of an individual siRNA strand of the siRNA duplex. The same purified siRNA provides three peaks when analyzed by capillary gel electrophoresis (CGE), one peak presumably corresponding to the duplex siRNA, and two peaks presumably corresponding to the separate siRNA sequence strands. Ion exchange HPLC analysis of the same siRNA contract only shows a single peak.

### Example 2

### Identification of Potential siRNA Target Sites in Any RNA Sequence

The sequence of an RNA target of interest, such as a viral or human MRNA transcript, is screened for target sites, for example by using a computer folding algorithm. In a non-limiting example, the sequence of a gene or RNA gene transcript derived from a database, such as Genbank, is used to generate siRNA targets having complimentarily to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. In a non-limiting example, anywhere from 1 to 1000 target sites are chosen within the transcript based on the size of the siRNA construct to be used. High throughput screening assays can be developed for screening siRNA molecules using methods known in the art, such as with multi-well or multi-plate assays to determine efficient reduction in target gene expression.

### Example 3

### Selection of siRNA Molecule Target Sites in a RNA

The following non-limiting steps can be used to carry out the selection of siRNAs targeting a given gene sequence or transcript.

1. The target sequence is parsed in silico into a list of all fragments or subsequences of a particular length, for example 23 nucleotide fragments, contained within the target sequence. This step is typically carried out using a custom Perl script, but commercial sequence analysis programs such as Oligo, MacVector, or the GCG Wisconsin Package can be employed as well.

2. In some instances the siRNAs correspond to more than one target sequence; such would be the case for example in targeting many different strains of a viral sequence, for targeting different transcripts of the same gene, targeting different transcripts of more than one gene, or for targeting both the human gene and an animal homolog. In this case, a subsequence list of a particular length is generated for each of the targets, and then the lists are compared to find matching sequences in each list. The subsequences are then ranked according to the number of target sequences that contain the given subsequence; the goal is to find subsequences that are present in most or all of the target sequences. Alternately, the ranking can indentify subsequences that are unique to a target sequence, such as a mutant target sequence. Such an approach would enable the use of siRNA to target specifically the mutant sequence and not effect the expression of the normal sequence.

3. In some instances the siRNA subsequences are absent in one or more sequences while present in the desired target sequence; such would be the case if the siRNA targets a gene with a paralogous family member that is to remain untargeted. As in case 2 above, a subsequence list of a particular length is generated for each of the targets, and then the lists are compared to find sequences that are present in the target gene but are absent in the untargeted paralog.

4. The ranked siRNA subsequences can be further analyzed and ranked according to GC content. A preference can be given to sites containing 30-70% GC, with a further preference to sites containing 40-60% GC.

5. The ranked siRNA subsequences can be further analyzed and ranked according to self-folding and internal hairpins. Weaker internal folds are preferred; strong hairpin structures are to be avoided.

6. The ranked siRNA subsequences can be further analyzed and ranked according to whether they have runs of GGG or CCC in the sequence. GGG (or even more Gs) in either strand can make oligonucleotide synthesis problematic, so it is avoided whenever better sequences are available. CCC is searched in the target strand because that will place GGG in the antisense strand.

7. The ranked siRNA subsequences can be further analyzed and ranked according to whether they have the dinucleotide UU (uridine dinucleotide) on the 3′ end of the sequence, and/or AA on the 5′ end of the sequence (to yield 3′ UU on the antisense sequence). These sequences allow one to design siRNA molecules with terminal TT thymidine dinucleotides.

8. Four or five target sites are chosen from the ranked list of subsequences as described above. For example, in subsequences having 23 nucleotides, the right 21 nucleotides of each chosen 23-mer subsequence are then designed and synthesized for the upper (sense) strand of the siRNA duplex, while the reverse complement of the left 21 nucleotides of each chosen 23-mer subsequence are then designed and synthesized for the lower (antisense) strand of the siRNA duplex. If terminal TT residues are desired for the sequence (as described in paragraph 7), then the two 3′ terminal nucleotides of both the sense and antisense strands are replaced by TT prior to synthesizing the oligos.

9. The siRNA molecules are screened in an in vitro, cell culture or animal model system to identify the most active siRNA molecule or the most preferred target site within the target RNA sequence.

### Example 4

### PTP-1B Targeted siRNA Design

siRNA target sites were chosen by analyzing sequences of the PTP-1B RNA target and optionally prioritizing the target sites on the basis of folding (structure of any given sequence analyzed to determine siRNA accessibility to the target). siRNA molecules were designed that could bind each target and are optionally individually analyzed by computer folding to assess whether the siRNA molecule can interact with the target sequence. Varying the length of the siRNA molecules can be chosen to optimize activity. Generally, a sufficient number of complementary nucleotide bases are chosen to bind to, or otherwise interact with, the target RNA, but the degree of complementarity can be modulated to accommodate siRNA duplexes or varying length or base composition. By using such methodologies, siRNA molecules can be designed to target sites within any known RNA sequence, for example those RNA sequences corresponding to the any gene transcript.

### Example 5

### Chemical Synthesis and Purification of siRNA

siRNA molecules can be designed to interact with various sites in the RNA message, for example target sequences within the RNA sequences described herein. The sequence of one strand of the siRNA molecule(s) are complementary to the target site sequences described above. The siRNA molecules can be chemically synthesized using methods described herein. Inactive siRNA molecules that are used as control sequences can be synthesized by scrambling the sequence of the siRNA molecules such that it is not complementary to the target sequence.

### Example 6

### RNAi In Vitro Assay to Assess siRNA Activity

An in vitro assay that recapitulates RNAi in a cell free system is used to evaluate siRNA constructs targeting PTP-1B RNA targets. The assay comprises the system described by Tuschl et al., 1999, Genes and Development, 13, 3191-3197 and Zamore et al., 2000, Cell, 101, 25-33 adapted for use with PTP-1B target RNA. A Drosophila extract derived from syncytial blastoderm is used to reconstitute RNAi activity in vitro. Target RNA is generated via in vitro transcription from an appropriate PTP-1B expressing plasmid using T7 RNA polymerase or via chemical synthesis as described herein. Sense and antisense siRNA strands (for example 20 uM each) are annealed by incubation in buffer (such as 100 mM potassium acetate, 30 mM HEPES-KOH, pH 7.4, 2 mM magnesium acetate) for 1 min. at 90° C. followed by 1 hour at 37° C., then diluted in lysis buffer (for example 100 mM potassium acetate, 30 mM HEPES-KOH at pH 7.4, 2 mM magnesium acetate). Annealing can be monitored by gel electrophoresis on an agarose gel in TBE buffer and stained with ethidium bromide. The Drosophila lysate is prepared using zero to two hour old embryos from Oregon R flies collected on yeasted molasses agar that are dechorionated and lysed. The lysate is centrifuged and the supernatant isolated. The assay comprises a reaction mixture containing 50% lysate [vol/vol], RNA (10-50 pM final concentration), and 10% [vol/vol] lysis buffer containing siRNA (10 nM final concentration). The reaction mixture also contains 10 mM creatine phosphate, 10 ug.ml creatine phosphokinase, 100 um GTP, 100 uM UTP, 100 uM CTP, 500 uM ATP, 5 mM DTT, 0.1 U/uL RNasin (Promega), and 100 uM of each amino acid. The final concentration of potassium acetate is adjusted to 100 mM. The reactions are pre-assembled on ice and preincubated at 25° C. for 10 minutes before adding RNA, then incubated at 25° C. for an additional 60 minutes. Reactions are quenched with 4 volumes of 1.25×Passive Lysis Buffer (Promega). Target RNA cleavage is assayed by RT-PCR analysis or other methods known in the art and are compared to control reactions in which siRNA is omitted from the reaction.

Alternately, internally-labeled target RNA for the assay is prepared by in vitro transcription in the presence of [a-³²p] CTP, passed over a G 50 Sephadex column by spin chromatography and used as target RNA without further purification. Optionally, target RNA is 5′-³²P-end labeled using T4 polynucleotide kinase enzyme. Assays are performed as described above and target RNA and the specific RNA cleavage products generated by RNAi are visualized on an autoradiograph of a gel. The percentage of cleavage is determined by Phosphor Imager® quantitation of bands representing intact control RNA or RNA from control reactions without siRNA and the cleavage products generated by the assay.

### Example 7

### Cell Culture Models

Various methods have been developed to assay PTP-1B activity in vitro and in vivo. Maegawa et al., 1995, J. Biol. Chem., 270, 7724-7730, describe a tissue culture model in which Rat 1 fibroblasts expressing human insulin receptors can be used to model hyperglycemia induced insulin resistance. Maegawa et al. also describe assays to measure PTPase activity using labeled Nature, 379, 840-844, describe a murine tissue culture model employing Gi□□ deficiency to study hyperinsulinaemia, impaired glucose tolerance and resistance to insulin in vivo. Assays for PTPase activity and tyrosine phosphorylation of insulin-receptor substrate 1 are also described. Wang et al., 1999, Biochim. Biophys. Acta, 1431, 14-23, describe fluorescein monophosphates as fluorogenic substrates for PTPs which can be used to study PTPase modulation. The use of such fluorogenic PTP-1B substrates could be used to develop a high throughput screening assay for siRNA-based inhibition of PTP-1B in vivo.

### Example 8

### Animal Models

Khandelwal et al., 1995, Molecular and Cellular Biochemistry, 153, 87-94, describe four different animal models for studying insulin dependent and insulin resistant diabetes mellitus. These models were used to study the effect of vanadate, an insulin mimetic and PTPase inhibitor, on the insulin-stimulated phosphorylation of the insulin receptor and its tyrosine kinase activity. Elchebly et al., 1999, Science, 283, 1544-1548, describe a murine PTP-1B knockout model in which insulin sensitivity and fuel metabolism are studied. The resulting PTP-1B deficient mice (both homozygous PTP-1B^{-/-} and heterozygous PTP-1B^{+/-}) were healthy and, in the fed state, had lower blood glucose and circulating insulin levels that were one-half that of their PTP-1B^{+/+} expressing littermates. These PTP-1B deficient mice demonstrated enhanced insulin sensitivity in glucose and insulin tolerance tests. At the physiological level, the PTP-1B deficient mice showed increased phosphorylation of the insulin receptor after insulin administration. When fed a high fat diet, the PTP-1B deficient mice were resistant to weight gain and remained insulin sensitive as opposed to normal PTP-1B expressing mice, who rapidly gained weight and become insulin resistant.

### Indications

Particular degenerative and disease states that can be associated with PTP-1B expression modulation include but are not limited to:

3. Diabetes: Both type 1 and type 2 diabetes may be treated by modulation of PTP-1B expression. Type 2 diabetes correlates to desensitized insulin receptor function (White et al., 1994). Disruption of the PTP-1B dephosphorylation of the insulin receptor in vivo manifests in insulin sensitivity and increased insulin receptor autophosphorylation (Elchebly et al., 1999). Insulin dependant diabetes, type 1, may respond to PTP-1B modulation through increased insulin sensitivity.

4. Obesity: Elchebly et al., 1999, demonstrated that PTP-1B deficient mice were resistant to weight gain when fed a high fat diet compared to normal PTP-1B expressing mice. This finding suggests that PTP-1B modulation may be beneficial in the treatment of obesity. Ahmad et al., 1997, Metab. Clin. Exp., 46, 1140-1145, describe reduced PTPs in adipose tissue and improved insulin sensitivity in obese subjects following weight loss.

The present body of knowledge in PTP-1B research indicates the need for methods to assay PTP-1B activity and for compounds that can regulate PTP-1B expression for research, diagnostic, and therapeutic use.

Troglitazone is a non-limiting example of a pharmaceutical agent that can be combined with or used in conjunction with the nucleic acid molecules (e.g. siRNA molecules) of the instant invention. Those skilled in the art will recognize that other drugs such as anti-diabetes and anti-obesity compounds and therapies can be similarly be readily combined with the nucleic acid molecules of the instant invention (e.g. siRNA molecules) are hence within the scope of the instant invention.

### Diagnostic Uses

The siRNA molecules of the invention can be used in a variety of diagnostic applications, such as in identifying molecular targets such as RNA in a variety of applications, for example, in clinical, industrial, environmental, agricultural and/or research settings. Such diagnostic use of siRNA molecules involves utilizing reconstituted RNAi systems, for example using cellular lysates or partially purified cellular lysates. siRNA molecules of this invention may be used as diagnostic tools to examine genetic drift and mutations within diseased cells or to detect the presence of endogenous or exogenous, for example viral, RNA in a cell. The close relationship between siRNA activity and the structure of the target RNA allows the detection of mutations in any region of the molecule, which alters the base-pairing and three-dimensional structure of the target RNA. By using multiple siRNA molecules described in this invention, one may map nucleotide changes, which are important to RNA structure and function in vitro, as well as in cells and tissues. Cleavage of target RNAs with siRNA molecules can be used to inhibit gene expression and define the role (essentially) of specified gene products in the progression of disease or infection. In this manner, other genetic targets may be defined as important mediators of the disease. These experiments will lead to better treatment of the disease progression by affording the possibility of combination therapies (e.g., multiple siRNA molecules targeted to different genes, siRNA molecules coupled with known small molecule inhibitors, or intermittent treatment with combinations siRNA molecules and/or other chemical or biological molecules). Other in vitro uses of siRNA molecules of this invention are well known in the art, and include detection of the presence of mRNAs associated with a disease, infection, or related condition. Such RNA is detected by determining the presence of a cleavage product after treatment with a siRNA using standard methodologies, for example fluorescence resonance emission transfer (FRET).

In a specific example, siRNA molecules that can cleave only wild-type or mutant forms of the target RNA are used for the assay. The first siRNA molecules is used to identify wild-type RNA present in the sample and the second siRNA molecules will be used to identify mutant RNA in the sample. As reaction controls, synthetic substrates of both wild-type and mutant RNA will be cleaved by both siRNA molecules to demonstrate the relative siRNA efficiencies in the reactions and the absence of cleavage of the “non-targeted” RNA species. The cleavage products from the synthetic substrates will also serve to generate size markers for the analysis of wild-type and mutant RNAs in the sample population. Thus each analysis will require two siRNA molecules, two substrates and one unknown sample which will be combined into six reactions. The presence of cleavage products will be determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., disease related or infection related) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations that are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE 1
PTP-1B target and siRNA sequences

| | | Seq | | | Seq | | | Seq |
|---|---|---|---|---|---|---|---|---|
| Pos | Target Sequence | ID | UPos | Upper seq | ID | LPos | Lower seq | ID |
| 1 | GUGAUGCGUAGUUCCGGCU | 1 | 1 | GUGAUGCGUAGUUCCGGCU | 1 | 23 | AGCCGGAACUACGCAUCAC | 186 |
| 19 | UGCCGGUUGACAUGAAGAA | 2 | 19 | UGCCGGUUGACAUGAAGAA | 2 | 41 | UUCUUCAUGUCAACCGGCA | 187 |
| 37 | AGCAGCAGCGGCUAGGGCG | 3 | 37 | AGCAGCAGCGGCUAGGGCG | 3 | 59 | CGCCCUAGCCGCUGCUGCU | 188 |
| 55 | GGCGGUAGCUGCAGGGGUC | 4 | 55 | GGCGGUAGCUGCAGGGGUC | 4 | 77 | GACCCCUGCAGCUACCGCC | 189 |
| 73 | CGGGGAUUGCAGCGGGCCU | 5 | 73 | CGGGGAUUGCAGCGGGCCU | 5 | 95 | AGGCCCGCUGCAAUCCCCG | 190 |
| 91 | UCGGGGCUAAGAGCGCGAC | 6 | 91 | UCGGGGCUAAGAGCGCGAC | 6 | 113 | GUCGCGCUCUUAGCCCCGA | 191 |
| 109 | CGCGGCCUAGAGCGGCAGA | 7 | 109 | CGCGGCCUAGAGCGGCAGA | 7 | 131 | UCUGCCGCUCUAGGCCGCG | 192 |
| 127 | ACGGCGCAGUGGGCCGAGA | 8 | 127 | ACGGCGCAGUGGGCCGAGA | 8 | 149 | UCUCGGCCCACUGCGCCGU | 193 |
| 145 | AAGGAGGCGCAGCAGCCGC | 9 | 145 | AAGGAGGCGCAGCAGCCGC | 9 | 167 | GCGGCUGCUGCGCCUCCUU | 194 |
| 163 | CCCUGGCCCGUCAUGGAGA | 10 | 163 | CCCUGGCCCGUCAUGGAGA | 10 | 185 | UCUCCAUGACGGGCCAGGG | 195 |
| 181 | AUGGAAAAGGAGUUCGAGC | 11 | 181 | AUGGAAAAGGAGUUCGAGC | 11 | 203 | GCUCGAACUCCUUUUCCAU | 196 |
| 199 | CAGAUCGACAAGUCCGGGA | 12 | 199 | CAGAUCGACAAGUCCGGGA | 12 | 221 | UCCCGGACUUGUCGAUCUG | 197 |
| 217 | AGCUGGGCGGCCAUUUACC | 13 | 217 | AGCUGGGCGGCCAUUUACC | 13 | 239 | GGUAAAUGGCCGCCCAGCU | 198 |
| 235 | CAGGAUAUCCGACAUGAAG | 14 | 235 | CAGGAUAUCCGACAUGAAG | 14 | 257 | CUUCAUGUCGGAUAUCCUG | 199 |
| 253 | GCCAGUGACUUCCCAUGUA | 15 | 253 | GCCAGUGACUUCCCAUGUA | 15 | 275 | UACAUGGGAAGUCACUGGC | 200 |
| 271 | AGAGUGGCCAAGCUUCCUA | 16 | 271 | AGAGUGGCCAAGCUUCCUA | 16 | 293 | UAGGAAGCUUGGCCACUCU | 201 |
| 289 | AAGAACAAAAACCGAAAUA | 17 | 289 | AAGAACAAAAACCGAAAUA | 17 | 311 | UAUUUCGGUUUUUGUUCUU | 202 |
| 307 | AGGUACAGAGACGUCAGUC | 18 | 307 | AGGUACAGAGACGUCAGUC | 18 | 329 | GACUGACGUCUCUGUACCU | 203 |
| 325 | CCCUUUGACCAUAGUCGGA | 19 | 325 | CCCUUUGACCAUAGUCGGA | 19 | 347 | UCCGACUAUGGUCAAAGGG | 204 |
| 343 | AUUAAACUACAUCAAGAAG | 20 | 343 | AUUAAACUACAUCAAGAAG | 20 | 365 | CUUCUUGAUGUAGUUUAAU | 205 |
| 361 | GAUAAUGACUAUAUCAACG | 21 | 361 | GAUAAUGACUAUAUCAACG | 21 | 383 | CGUUGAUAUAGUCAUUAUC | 206 |
| 379 | GCUAGUUUGAUAAAAAUGG | 22 | 379 | GCUAGUUUGAUAAAAAUGG | 22 | 401 | CCAUUUUUAUCAAACUAGC | 207 |
| 397 | GAAGAAGCCCAAAGGAGUU | 23 | 397 | GAAGAAGCCCAAAGGAGUU | 23 | 419 | AACUCCUUUGGGCUUCUUC | 208 |
| 415 | UACAUUCUUACCCAGGGCC | 24 | 415 | UACAUUCUUACCCAGGGCC | 24 | 437 | GGCCCUGGGUAAGAAUGUA | 209 |
| 433 | CCUUUGCCUAACACAUGCG | 25 | 433 | CCUUUGCCUAACACAUGCG | 25 | 455 | CGCAUGUGUUAGGCAAAGG | 210 |
| 451 | GGUCACUUUUGGGAGAUGG | 26 | 451 | GGUCACUUUUGGGAGAUGG | 26 | 473 | CCAUCUCCCAAAAGUGACC | 211 |
| 469 | GUGUGGGAGCAGAAAAGCA | 27 | 469 | GUGUGGGAGCAGAAAAGCA | 27 | 491 | UGCUUUUCUGCUCCCACAC | 212 |
| 487 | AGGGGUGUCGUCAUGCUCA | 28 | 487 | AGGGGUGUCGUCAUGCUCA | 28 | 509 | UGAGCAUGACGACACCCCU | 213 |
| 505 | AACAGAGUGAUGGAGAAAG | 29 | 505 | AACAGAGUGAUGGAGAAAG | 29 | 527 | CUUUCUCCAUCACUCUGUU | 214 |
| 523 | GGUUCGUUAAAAUGCGCAC | 30 | 523 | GGUUCGUUAAAAUGCGCAC | 30 | 545 | GUGCGCAUUUUAACGAACC | 215 |
| 541 | CAAUACUGGCCACAAAAAG | 31 | 541 | CAAUACUGGCCACAAAAAG | 31 | 563 | CUUUUUGUGGCCAGUAUUG | 216 |
| 559 | GAAGAAAAAGAGAUGAUCU | 32 | 559 | GAAGAAAAAGAGAUGAUCU | 32 | 581 | AGAUCAUCUCUUUUUCUUC | 217 |
| 577 | UUUGAAGACACAAAUUUGA | 33 | 577 | UUUGAAGACACAAAUUUGA | 33 | 599 | UCAAAUUUGUGUCUUCAAA | 218 |
| 595 | AAAUUAACAUUGAUCUCUG | 34 | 595 | AAAUUAACAUUGAUCUCUG | 34 | 617 | CAGAGAUCAAUGUUAAUUU | 219 |
| 613 | GAAGAUAUCAAGUCAUAUU | 35 | 613 | GAAGAUAUCAAGUCAUAUU | 35 | 635 | AAUAUGACUUGAUAUCUUC | 220 |
| 631 | UAUACAGUGCGACAGCUAG | 36 | 631 | UAUACAGUGCGACAGCUAG | 36 | 653 | CUAGCUGUCGCACUGUAUA | 221 |
| 649 | GAAUUGGAAAACCUUACAA | 37 | 649 | GAAUUGGAAAACCUUACAA | 37 | 671 | UUGUAAGGUUUUCCAAUUC | 222 |
| 667 | ACCCAAGAAACUCGAGAGA | 38 | 667 | ACCCAAGAAACUCGAGAGA | 38 | 689 | UCUCUCGAGUUUCUUGGGU | 223 |
| 685 | AUCUUACAUUUCCACUAUA | 39 | 685 | AUCUUACAUUUCCACUAUA | 39 | 707 | UAUAGUGGAAAUGUAAGAU | 224 |
| 703 | ACCACAUGGCCUGACUUUG | 40 | 703 | ACCACAUGGCCUGACUUUG | 40 | 725 | CAAAGUCAGGCCAUGUGGU | 225 |
| 721 | GGAGUCCCUGAAUCACCAG | 41 | 721 | GGAGUCCCUGAAUCACCAG | 41 | 743 | CUGGUGAUUCAGGGACUCC | 226 |
| 739 | GCCUCAUUCUUGAACUUUC | 42 | 739 | GCCUCAUUCUUGAACUUUC | 42 | 761 | GAAAGUUCAAGAAUGAGGC | 227 |
| 757 | CUUUUCAAAGUCCGAGAGU | 43 | 757 | CUUUUCAAAGUCCGAGAGU | 43 | 779 | ACUCUCGGACUUUGAAAAG | 228 |
| 775 | UCAGGGUCACUCAGCCCGG | 44 | 775 | UCAGGGUCACUCAGCCCGG | 44 | 797 | CCGGGCUGAGUGACCCUGA | 229 |
| 793 | GAGCACGGGCCCGUUGUGG | 45 | 793 | GAGCACGGGCCCGUUGUGG | 45 | 815 | CCACAACGGGCCCGUGCUC | 230 |
| 811 | GUGCACUGCAGUGCAGGCA | 46 | 811 | GUGCACUGCAGUGCAGGCA | 46 | 833 | UGCCUGCACUGCAGUGCAC | 231 |
| 829 | AUCGGCAGGUCUGGAACCU | 47 | 829 | AUCGGCAGGUCUGGAACCU | 47 | 851 | AGGUUCCAGACCUGCCGAU | 232 |
| 847 | UUCUGUCUGGCUGAUACCU | 48 | 847 | UUCUGUCUGGCUGAUACCU | 48 | 869 | AGGUAUCAGCCAGACAGAA | 233 |
| 865 | UGCCUCUUGCUGAUGGACA | 49 | 865 | UGCCUCUUGCUGAUGGACA | 49 | 887 | UGUCCAUCAGCAAGAGGCA | 234 |
| 883 | AAGAGGAAAGACCCUUCUU | 50 | 883 | AAGAGGAAAGACCCUUCUU | 50 | 905 | AAGAAGGGUCUUUCCUCUU | 235 |
| 901 | UCCGUUGAUAUCAAGAAAG | 51 | 901 | UCCGUUGAUAUCAAGAAAG | 51 | 923 | CUUUCUUGAUAUCAACGGA | 236 |
| 919 | GUGCUGUUAGAAAUGAGGA | 52 | 919 | GUGCUGUUAGAAAUGAGGA | 52 | 941 | UCCUCAUUUCUAACAGCAC | 237 |
| 937 | AAGUUUCGGAUGGGGCUGA | 53 | 937 | AAGUUUCGGAUGGGGCUGA | 53 | 959 | UCAGCCCCAUCCGAAACUU | 238 |
| 955 | AUCCAGACAGCCGACCAGC | 54 | 955 | AUCCAGACAGCCGACCAGC | 54 | 977 | GCUGGUCGGCUGUCUGGAU | 239 |
| 973 | CUGCGCUUCUCCUACCUGG | 55 | 973 | CUGCGCUUCUCCUACCUGG | 55 | 995 | CCAGGUAGGAGAAGCGCAG | 240 |
| 991 | GCUGUGAUCGAAGGUGCCA | 56 | 991 | GCUGUGAUCGAAGGUGCCA | 56 | 1013 | UGGCACCUUCGAUCACAGC | 241 |
| 1009 | AAAUUCAUCAUGGGGGACU | 57 | 1009 | AAAUUCAUCAUGGGGGACU | 57 | 1031 | AGUCCCCCAUGAUGAAUUU | 242 |
| 1027 | UCUUCCGUGCAGGAUCAGU | 58 | 1027 | UCUUCCGUGCAGGAUCAGU | 58 | 1049 | ACUGAUCCUGCACGGAAGA | 243 |
| 1045 | UGGAAGGAGCUUUCCCACG | 59 | 1045 | UGGAAGGAGCUUUCCCACG | 59 | 1067 | CGUGGGAAAGCUCCUUCCA | 244 |
| 1063 | GAGGACCUGGAGCCCCCAC | 60 | 1063 | GAGGACCUGGAGCCCCCAC | 60 | 1085 | GUGGGGGCUCCAGGUCCUC | 245 |
| 1081 | CCCGAGCAUAUCCCCCCAC | 61 | 1081 | CCCGAGCAUAUCCCCCCAC | 61 | 1103 | GUGGGGGGAUAUGCUCGGG | 246 |
| 1099 | CCUCCCCGGCCACCCAAAC | 62 | 1099 | CCUCCCCGGCCACCCAAAC | 62 | 1121 | GUUUGGGUGGCCGGGGAGG | 247 |
| 1117 | CGAAUCCUGGAGCCACACA | 63 | 1117 | CGAAUCCUGGAGCCACACA | 63 | 1139 | UGUGUGGCUCCAGGAUUCG | 248 |
| 1135 | AAUGGGAAAUGCAGGGAGU | 64 | 1135 | AAUGGGAAAUGCAGGGAGU | 64 | 1157 | ACUCCCUGCAUUUCCCAUU | 249 |
| 1153 | UUCUUCCCAAAUCACCAGU | 65 | 1153 | UUCUUCCCAAAUCACCAGU | 65 | 1175 | ACUGGUGAUUUGGGAAGAA | 250 |
| 1171 | UGGGUGAAGGAAGAGACCC | 66 | 1171 | UGGGUGAAGGAAGAGACCC | 66 | 1193 | GGGUCUCUUCCUUCACCCA | 251 |
| 1189 | CAGGAGGAUAAAGACUGCC | 67 | 1189 | CAGGAGGAUAAAGACUGCC | 67 | 1211 | GGCAGUCUUUAUCCUCCUG | 252 |
| 1207 | CCCAUCAAGGAAGAAAAAG | 68 | 1207 | CCCAUCAAGGAAGAAAAAG | 68 | 1229 | CUUUUUCUUCCUUGAUGGG | 253 |
| 1225 | GGAAGCCCCUUAAAUGCCG | 69 | 1225 | GGAAGCCCCUUAAAUGCCG | 69 | 1247 | CGGCAUUUAAGGGGCUUCC | 254 |
| 1243 | GCACCCUACGGCAUCGAAA | 70 | 1243 | GCACCCUACGGCAUCGAAA | 70 | 1265 | UUUCGAUGCCGUAGGGUGC | 255 |
| 1261 | AGCAUGAGUCAAGACACUG | 71 | 1261 | AGCAUGAGUCAAGACACUG | 71 | 1283 | CAGUGUCUUGACUCAUGCU | 256 |
| 1279 | GAAGUUAGAAGUCGGGUCG | 72 | 1279 | GAAGUUAGAAGUCGGGUCG | 72 | 1301 | CGACCCGACUUCUAACUUC | 257 |
| 1297 | GUGGGGGGAAGUCUUCGAG | 73 | 1297 | GUGGGGGGAAGUCUUCGAG | 73 | 1319 | CUCGAAGACUUCCCCCCAC | 258 |
| 1315 | GGUGCCCAGGCUGCCUCCC | 74 | 1315 | GGUGCCCAGGCUGCCUCCC | 74 | 1337 | GGGAGGCAGCCUGGGCACC | 259 |
| 1333 | CCAGCCAAAGGGGAGCCGU | 75 | 1333 | CCAGCCAAAGGGGAGCCGU | 75 | 1355 | ACGGCUCCCCUUUGGCUGG | 260 |
| 1351 | UCACUGCCCGAGAAGGACG | 76 | 1351 | UCACUGCCCGAGAAGGACG | 76 | 1373 | CGUCCUUCUCGGGCAGUGA | 261 |
| 1369 | GAGGACCAUGCACUGAGUU | 77 | 1369 | GAGGACCAUGCACUGAGUU | 77 | 1391 | AACUCAGUGCAUGGUCCUC | 262 |
| 1387 | UACUGGAAGCCCUUCCUGG | 78 | 1387 | UACUGGAAGCCCUUCCUGG | 78 | 1409 | CCAGGAAGGGCUUCCAGUA | 263 |
| 1405 | GUCAACAUGUGCGUGGCUA | 79 | 1405 | GUCAACAUGUGCGUGGCUA | 79 | 1427 | UAGCCACGCACAUGUUGAC | 264 |
| 1423 | ACGGUCCUCACGGCCGGCG | 80 | 1423 | ACGGUCCUCACGGCCGGCG | 80 | 1445 | CGCCGGCCGUGAGGACCGU | 265 |
| 1441 | GCUUACCUCUGCUACAGGU | 81 | 1441 | GCUUACCUCUGCUACAGGU | 81 | 1463 | ACCUGUAGCAGAGGUAAGC | 266 |
| 1459 | UUCCUGUUCAACAGCAACA | 82 | 1459 | UUCCUGUUCAACAGCAACA | 82 | 1481 | UGUUGCUGUUGAACAGGAA | 267 |
| 1477 | ACAUAGCCUGACCCUCCUC | 83 | 1477 | ACAUAGCCUGACCCUCCUC | 83 | 1499 | GAGGAGGGUCAGGCUAUGU | 268 |
| 1495 | CCACUCCACCUCCACCCAC | 84 | 1495 | CCACUCCACCUCCACCCAC | 84 | 1517 | GUGGGUGGAGGUGGAGUGG | 269 |
| 1513 | CUGUCCGCCUCUGCCCGCA | 85 | 1513 | CUGUCCGCCUCUGCCCGCA | 85 | 1535 | UGCGGGCAGAGGCGGACAG | 270 |
| 1531 | AGAGCCCACGCCCGACUAG | 86 | 1531 | AGAGCCCACGCCCGACUAG | 86 | 1553 | CUAGUCGGGCGUGGGCUCU | 271 |
| 1549 | GCAGGCAUGCCGCGGUAGG | 87 | 1549 | GCAGGCAUGCCGCGGUAGG | 87 | 1571 | CCUACCGCGGCAUGCCUGC | 272 |
| 1567 | GUAAGGGCCGCCGGACCGC | 88 | 1567 | GUAAGGGCCGCCGGACCGC | 88 | 1589 | GCGGUCCGGCGGCCCUUAC | 273 |
| 1585 | CGUAGAGAGCCGGGCCCCG | 89 | 1585 | CGUAGAGAGCCGGGCCCCG | 89 | 1607 | CGGGGCCCGGCUCUCUACG | 274 |
| 1603 | GGACGGACGUUGGUUCUGC | 90 | 1603 | GGACGGACGUUGGUUCUGC | 90 | 1625 | GCAGAACCAACGUCCGUCC | 275 |
| 1621 | CACUAAAACCCAUCUUCCC | 91 | 1621 | CACUAAAACCCAUCUUCCC | 91 | 1643 | GGGAAGAUGGGUUUUAGUG | 276 |
| 1639 | CCGGAUGUGUGUCUCACCC | 92 | 1639 | CCGGAUGUGUGUCUCACCC | 92 | 1661 | GGGUGAGACACACAUCCGG | 277 |
| 1657 | CCUCAUCCUUUUACUUUUU | 93 | 1657 | CCUCAUCCUUUUACUUUUU | 93 | 1679 | AAAAAGUAAAAGGAUGAGG | 278 |
| 1675 | UGCCCCUUCCACUUUGAGU | 94 | 1675 | UGCCCCUUCCACUUUGAGU | 94 | 1697 | ACUCAAAGUGGAAGGGGCA | 279 |
| 1693 | UACCAAAUCCACAAGCCAU | 95 | 1693 | UACCAAAUCCACAAGCCAU | 95 | 1715 | AUGGCUUGUGGAUUUGGUA | 280 |
| 1711 | UUUUUUGAGGAGAGUGAAA | 96 | 1711 | UUUUUUGAGGAGAGUGAAA | 96 | 1733 | UUUCACUCUCCUCAAAAAA | 281 |
| 1729 | AGAGAGUACCAUGCUGGCG | 97 | 1729 | AGAGAGUACCAUGCUGGCG | 97 | 1751 | CGCCAGCAUGGUACUCUCU | 282 |
| 1747 | GGCGCAGAGGGAAGGGGCC | 98 | 1747 | GGCGCAGAGGGAAGGGGCC | 98 | 1769 | GGCCCCUUCCCUCUGCGCC | 283 |
| 1765 | CUACACCCGUCUUGGGGCU | 99 | 1765 | CUACACCCGUCUUGGGGCU | 99 | 1787 | AGCCCCAAGACGGGUGUAG | 284 |
| 1783 | UCGCCCCACCCAGGGCUCC | 100 | 1783 | UCGCCCCACCCAGGGCUCC | 100 | 1805 | GGAGCCCUGGGUGGGGCGA | 285 |
| 1801 | CCUCCUGGAGCAUCCCAGG | 101 | 1801 | CCUCCUGGAGCAUCCCAGG | 101 | 1823 | CCUGGGAUGCUCCAGGAGG | 286 |
| 1819 | GCGGGCGGCACGCCAACAG | 102 | 1819 | GCGGGCGGCACGCCAACAG | 102 | 1841 | CUGUUGGCGUGCCGCCCGC | 287 |
| 1837 | GCCCCCCCCUUGAAUCUGC | 103 | 1837 | GCCCCCCCCUUGAAUCUGC | 103 | 1859 | GCAGAUUCAAGGGGGGGGC | 288 |
| 1855 | CAGGGAGCAACUCUCCACU | 104 | 1855 | CAGGGAGCAACUCUCCACU | 104 | 1877 | AGUGGAGAGUUGCUCCCUG | 289 |
| 1873 | UCCAUAUUUAUUUAAACAA | 105 | 1873 | UCCAUAUUUAUUUAAACAA | 105 | 1895 | UUGUUUAAAUAAAUAUGGA | 290 |
| 1891 | AUUUUUUCCCCAAAGGCAU | 106 | 1891 | AUUUUUUCCCCAAAGGCAU | 106 | 1913 | AUGCCUUUGGGGAAAAAAU | 291 |
| 1909 | UCCAUAGUGCACUAGCAUU | 107 | 1909 | UCCAUAGUGCACUAGCAUU | 107 | 1931 | AAUGCUAGUGCACUAUGGA | 292 |
| 1927 | UUUCUUGAACCAAUAAUGU | 108 | 1927 | UUUCUUGAACCAAUAAUGU | 108 | 1949 | ACAUUAUUGGUUCAAGAAA | 293 |
| 1945 | UAUUAAAAUUUUUUGAUGU | 109 | 1945 | UAUUAAAAUUUUUUGAUGU | 109 | 1967 | ACAUCAAAAAAUUUUAAUA | 294 |
| 1963 | UCAGCCUUGCAUCAAGGGC | 110 | 1963 | UCAGCCUUGCAUCAAGGGC | 110 | 1985 | GCCCUUGAUGCAAGGCUGA | 295 |
| 1981 | CUUUAUCAAAAAGUACAAU | 111 | 1981 | CUUUAUCAAAAAGUACAAU | 111 | 2003 | AUUGUACUUUUUGAUAAAG | 296 |
| 1999 | UAAUAAAUCCUCAGGUAGU | 112 | 1999 | UAAUAAAUCCUCAGGUAGU | 112 | 2021 | ACUACCUGAGGAUUUAUUA | 297 |
| 2017 | UACUGGGAAUGGAAGGCUU | 113 | 2017 | UACUGGGAAUGGAAGGCUU | 113 | 2039 | AAGCCUUCCAUUCCCAGUA | 298 |
| 2035 | UUGCCAUGGGCCUGCUGCG | 114 | 2035 | UUGCCAUGGGCCUGCUGCG | 114 | 2057 | CGCAGCAGGCCCAUGGCAA | 299 |
| 2053 | GUCAGACCAGUACUGGGAA | 115 | 2053 | GUCAGACCAGUACUGGGAA | 115 | 2075 | UUCCCAGUACUGGUCUGAC | 300 |
| 2071 | AGGAGGACGGUUGUAAGCA | 116 | 2071 | AGGAGGACGGUUGUAAGCA | 116 | 2093 | UGCUUACAACCGUCCUCCU | 301 |
| 2089 | AGUUGUUAUUUAGUGAUAU | 117 | 2089 | AGUUGUUAUUUAGUGAUAU | 117 | 2111 | AUAUCACUAAAUAACAACU | 302 |
| 2107 | UUGUGGGUAACGUGAGAAG | 118 | 2107 | UUGUGGGUAACGUGAGAAG | 118 | 2129 | CUUCUCACGUUACCCACAA | 303 |
| 2125 | GAUAGAACAAUGCUAUAAU | 119 | 2125 | GAUAGAACAAUGCUAUAAU | 119 | 2147 | AUUAUAGCAUUGUUCUAUC | 304 |
| 2143 | UAUAUAAUGAACACGUGGG | 120 | 2143 | UAUAUAAUGAACACGUGGG | 120 | 2165 | CCCACGUGUUCAUUAUAUA | 305 |
| 2161 | GUAUUUAAUAAGAAACAUG | 121 | 2161 | GUAUUUAAUAAGAAACAUG | 121 | 2183 | CAUGUUUCUUAUUAAAUAC | 306 |
| 2179 | GAUGUGAGAUUACUUUGUC | 122 | 2179 | GAUGUGAGAUUACUUUGUC | 122 | 2201 | GACAAAGUAAUCUCACAUC | 307 |
| 2197 | CCCGCUUAUUCUCCUCCCU | 123 | 2197 | CCCGCUUAUUCUCCUCCCU | 123 | 2219 | AGGGAGGAGAAUAAGCGGG | 308 |
| 2215 | UGUUAUCUGCUAGAUCUAG | 124 | 2215 | UGUUAUCUGCUAGAUCUAG | 124 | 2237 | CUAGAUCUAGCAGAUAACA | 309 |
| 2233 | GUUCUCAAUCACUGCUCCC | 125 | 2233 | GUUCUCAAUCACUGCUCCC | 125 | 2255 | GGGAGCAGUGAUUGAGAAC | 310 |
| 2251 | CCCGUGUGUAUUAGAAUGC | 126 | 2251 | CCCGUGUGUAUUAGAAUGC | 126 | 2273 | GCAUUCUAAUACACACGGG | 311 |
| 2269 | CAUGUAAGGUCUUCUUGUG | 127 | 2269 | CAUGUAAGGUCUUCUUGUG | 127 | 2291 | CACAAGAAGACCUUACAUG | 312 |
| 2287 | GUCCUGAUGAAAAAUAUGU | 128 | 2287 | GUCCUGAUGAAAAAUAUGU | 128 | 2309 | ACAUAUUUUUCAUCAGGAC | 313 |
| 2305 | UGCUUGAAAUGAGAAACUU | 129 | 2305 | UGCUUGAAAUGAGAAACUU | 129 | 2327 | AAGUUUCUCAUUUCAAGCA | 314 |
| 2323 | UUGAUCUCUGCUUACUAAU | 130 | 2323 | UUGAUCUCUGCUUACUAAU | 130 | 2345 | AUUAGUAAGCAGAGAUCAA | 315 |
| 2341 | UGUGCCCCAUGUCCAAGUC | 131 | 2341 | UGUGCCCCAUGUCCAAGUC | 131 | 2363 | GACUUGGACAUGGGGCACA | 316 |
| 2359 | CCAACCUGCCUGUGCAUGA | 132 | 2359 | CCAACCUGCCUGUGCAUGA | 132 | 2381 | UCAUGCACAGGCAGGUUGG | 317 |
| 2377 | ACCUGAUCAUUACAUGGCU | 133 | 2377 | ACCUGAUCAUUACAUGGCU | 133 | 2399 | AGCCAUGUAAUGAUCAGGU | 318 |
| 2395 | UGUGGUUCCUAAGCCUGUU | 134 | 2395 | UGUGGUUCCUAAGCCUGUU | 134 | 2417 | AACAGGCUUAGGAACCACA | 319 |
| 2413 | UGCUGAAGUCAUUGUCGCU | 135 | 2413 | UGCUGAAGUCAUUGUCGCU | 135 | 2435 | AGCGACAAUGACUUCAGCA | 320 |
| 2431 | UCAGCAAUAGGGUGCAGUU | 136 | 2431 | UCAGCAAUAGGGUGCAGUU | 136 | 2453 | AACUGCACCCUAUUGCUGA | 321 |
| 2449 | UUUCCAGGAAUAGGCAUUU | 137 | 2449 | UUUCCAGGAAUAGGCAUUU | 137 | 2471 | AAAUGCCUAUUCCUGGAAA | 322 |
| 2467 | UGCCUAAUUCCUGGCAUGA | 138 | 2467 | UGCCUAAUUCCUGGCAUGA | 138 | 2489 | UCAUGCCAGGAAUUAGGCA | 323 |
| 2485 | ACACUCUAGUGACUUCCUG | 139 | 2485 | ACACUCUAGUGACUUCCUG | 139 | 2507 | CAGGAAGUCACUAGAGUGU | 324 |
| 2503 | GGUGAGGCCCAGCCUGUCC | 140 | 2503 | GGUGAGGCCCAGCCUGUCC | 140 | 2525 | GGACAGGCUGGGCCUCACC | 325 |
| 2521 | CUGGUACAGCAGGGUCUUG | 141 | 2521 | CUGGUACAGCAGGGUCUUG | 141 | 2543 | CAAGACCCUGCUGUACCAG | 326 |
| 2539 | GCUGUAACUCAGACAUUCC | 142 | 2539 | GCUGUAACUCAGACAUUCC | 142 | 2561 | GGAAUGUCUGAGUUACAGC | 327 |
| 2557 | CAAGGGUAUGGGAAGCCAU | 143 | 2557 | CAAGGGUAUGGGAAGCCAU | 143 | 2579 | AUGGCUUCCCAUACCCUUG | 328 |
| 2575 | UAUUCACACCUCACGCUCU | 144 | 2575 | UAUUCACACCUCACGCUCU | 144 | 2597 | AGAGCGUGAGGUGUGAAUA | 329 |
| 2593 | UGGACAUGAUUUAGGGAAG | 145 | 2593 | UGGACAUGAUUUAGGGAAG | 145 | 2615 | CUUCCCUAAAUCAUGUCCA | 330 |
| 2611 | GCAGGGACACCCCCCGCCC | 146 | 2611 | GCAGGGACACCCCCCGCCC | 146 | 2633 | GGGCGGGGGGUGUCCCUGC | 331 |
| 2629 | CCCCACCUUUGGGAUCAGC | 147 | 2629 | CCCCACCUUUGGGAUCAGC | 147 | 2651 | GCUGAUCCCAAAGGUGGGG | 332 |
| 2647 | CCUCCGCCAUUCCAAGUCA | 148 | 2647 | CCUCCGCCAUUCCAAGUCA | 148 | 2669 | UGACUUGGAAUGGCGGAGG | 333 |
| 2665 | AACACUCUUCUUGAGCAGA | 149 | 2665 | AACACUCUUCUUGAGCAGA | 149 | 2687 | UCUGCUCAAGAAGAGUGUU | 334 |
| 2683 | ACCGUGAUUUGGAAGAGAG | 150 | 2683 | ACCGUGAUUUGGAAGAGAG | 150 | 2705 | CUCUCUUCCAAAUCACGGU | 335 |
| 2701 | GGCACCUGCUGGAAACCAC | 151 | 2701 | GGCACCUGCUGGAAACCAC | 151 | 2723 | GUGGUUUCCAGCAGGUGCC | 336 |
| 2719 | CACUUCUUGAAACAGCCUG | 152 | 2719 | CACUUCUUGAAACAGCCUG | 152 | 2741 | CAGGCUGUUUCAAGAAGUG | 337 |
| 2737 | GGGUGACGGUCCUUUAGGC | 153 | 2737 | GGGUGACGGUCCUUUAGGC | 153 | 2759 | GCCUAAAGGACCGUCACCC | 338 |
| 2755 | CAGCCUGCCGCCGUCUCUG | 154 | 2755 | CAGCCUGCCGCCGUCUCUG | 154 | 2777 | CAGAGACGGCGGCAGGCUG | 339 |
| 2773 | GUCCCGGUUCACCUUGCCG | 155 | 2773 | GUCCCGGUUCACCUUGCCG | 155 | 2795 | CGGCAAGGUGAACCGGGAC | 340 |
| 2791 | GAGAGAGGCGCGUCUGCCC | 156 | 2791 | GAGAGAGGCGCGUCUGCCC | 156 | 2813 | GGGCAGACGCGCCUCUCUC | 341 |
| 2809 | CCACCCUCAAACCCUGUGG | 157 | 2809 | CCACCCUCAAACCCUGUGG | 157 | 2831 | CCACAGGGUUUGAGGGUGG | 342 |
| 2827 | GGGCCUGAUGGUGCUCACG | 158 | 2827 | GGGCCUGAUGGUGCUCACG | 158 | 2849 | CGUGAGCACCAUCAGGCCC | 343 |
| 2845 | GACUCUUCCUGCAAAGGGA | 159 | 2845 | GACUCUUCCUGCAAAGGGA | 159 | 2867 | UCCCUUUGCAGGAAGAGUC | 344 |
| 2863 | AACUGAAGACCUCCACAUU | 160 | 2863 | AACUGAAGACCUCCACAUU | 160 | 2885 | AAUGUGGAGGUCUUCAGUU | 345 |
| 2881 | UAAGUGGCUUUUUAACAUG | 161 | 2881 | UAAGUGGCUUUUUAACAUG | 161 | 2903 | CAUGUUAAAAAGCCACUUA | 346 |
| 2899 | GAAAAACACGGCAGCUGUA | 162 | 2899 | GAAAAACACGGCAGCUGUA | 162 | 2921 | UACAGCUGCCGUGUUUUUC | 347 |
| 2917 | AGCUCCCGAGCUACUCUCU | 163 | 2917 | AGCUCCCGAGCUACUCUCU | 163 | 2939 | AGAGAGUAGCUCGGGAGCU | 348 |
| 2935 | UUGCCAGCAUUUUCACAUU | 164 | 2935 | UUGCCAGCAUUUUCACAUU | 164 | 2957 | AAUGUGAAAAUGCUGGCAA | 349 |
| 2953 | UUUGCCUUUCUCGUGGUAG | 165 | 2953 | UUUGCCUUUCUCGUGGUAG | 165 | 2975 | CUACCACGAGAAAGGCAAA | 350 |
| 2971 | GAAGCCAGUACAGAGAAAU | 166 | 2971 | GAAGCCAGUACAGAGAAAU | 166 | 2993 | AUUUCUCUGUACUGGCUUC | 351 |
| 2989 | UUCUGUGGUGGGAACAUUC | 167 | 2989 | UUCUGUGGUGGGAACAUUC | 167 | 3011 | GAAUGUUCCCACCACAGAA | 352 |
| 3007 | CGAGGUGUCACCCUGCAGA | 168 | 3007 | CGAGGUGUCACCCUGCAGA | 168 | 3029 | UCUGCAGGGUGACACCUCG | 353 |
| 3025 | AGCUAUGGUGAGGUGUGGA | 169 | 3025 | AGCUAUGGUGAGGUGUGGA | 169 | 3047 | UCCACACCUCACCAUAGCU | 354 |
| 3043 | AUAAGGCUUAGGUGCCAGG | 170 | 3043 | AUAAGGCUUAGGUGCCAGG | 170 | 3065 | CCUGGCACCUAAGCCUUAU | 355 |
| 3061 | GCUGUAAGCAUUCUGAGCU | 171 | 3061 | GCUGUAAGCAUUCUGAGCU | 171 | 3083 | AGCUCAGAAUGCUUACAGC | 356 |
| 3079 | UGGGCUUGUUGUUUUUAAG | 172 | 3079 | UGGGCUUGUUGUUUUUAAG | 172 | 3101 | CUUAAAAACAACAAGCCCA | 357 |
| 3097 | GUCCUGUAUAUGUAUGUAG | 173 | 3097 | GUCCUGUAUAUGUAUGUAG | 173 | 3119 | CUACAUACAUAUACAGGAC | 358 |
| 3115 | GUAGUUUGGGUGUGUAUAU | 174 | 3115 | GUAGUUUGGGUGUGUAUAU | 174 | 3137 | AUAUACACACCCAAACUAC | 359 |
| 3133 | UAUAGUAGCAUUUCAAAAU | 175 | 3133 | UAUAGUAGCAUUUCAAAAU | 175 | 3155 | AUUUUGAAAUGCUACUAUA | 360 |
| 3151 | UGGACGUACUGGUUUAACC | 176 | 3151 | UGGACGUACUGGUUUAACC | 176 | 3173 | GGUUAAACCAGUACGUCCA | 361 |
| 3169 | CUCCUAUCCUUGGAGAGCA | 177 | 3169 | CUCCUAUCCUUGGAGAGCA | 177 | 3191 | UGCUCUCCAAGGAUAGGAG | 362 |
| 3187 | AGCUGGCUCUCCACCUUGU | 178 | 3187 | AGCUGGCUCUCCACCUUGU | 178 | 3209 | ACAAGGUGGAGAGCCAGCU | 363 |
| 3205 | UUACACAUUAUGUUAGAGA | 179 | 3205 | UUACACAUUAUGUUAGAGA | 179 | 3227 | UCUCUAACAUAAUGUGUAA | 364 |
| 3223 | AGGUAGCGAGCUGCUCUGC | 180 | 3223 | AGGUAGCGAGCUGCUCUGC | 180 | 3245 | GCAGAGCAGCUCGCUACCU | 365 |
| 3241 | CUAUAUGCCUUAAGCCAAU | 181 | 3241 | CUAUAUGCCUUAAGCCAAU | 181 | 3263 | AUUGGCUUAAGGCAUAUAG | 366 |
| 3259 | UAUUUACUCAUCAGGUCAU | 182 | 3259 | UAUUUACUCAUCAGGUCAU | 182 | 3281 | AUGACCUGAUGAGUAAAUA | 367 |
| 3277 | UUAUUUUUUACAAUGGCCA | 183 | 3277 | UUAUUUUUUACAAUGGCCA | 183 | 3299 | UGGCCAUUGUAAAAAAUAA | 368 |
| 3295 | AUGGAAUAAACCAUUUUUA | 184 | 3295 | AUGGAAUAAACCAUUUUUA | 184 | 3317 | UAAAAAUGGUUUAUUCCAU | 369 |
| 3300 | AUAAACCAUUUUUACAAAA | 185 | 3300 | AUAAACCAUUUUUACAAAA | 185 | 3322 | UUUUGUAAAAAUGGUUUAU | 370 |

PTP-1B = NM_002827 (PTPN1)

The 3′-ends of the Upper sequence and the Lower sequence of the siRNA construct can include a overhang sequence, for example about 1, 2, 3, or 4 nucleotides in length, preferably 2 nucleotides in length, wherein the overhanging sequence of the lower sequence is optionally complementary to a portion of the target sequence. The upper sequence is also referred to as the sense strand, whereas the lower sequence is also referred to as the antisense strand.

TABLE II
A. 2.5 μmol Synthesis Cycle ABI 394 Instrument

| Reagent | Equivalents | Amount | | Wait Time* DNA | | Wait Time* 2′-O-methyl | | Wait Time* RNA | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents: DNA/ | Amount: DNA/2′-O- | | | | Wait Time* 2′-O- | | | |
|---|---|---|---|---|---|---|---|---|---|
| Reagent | 2′-O-methyl/Ribo | methyl/Ribo | | Wait Time* DNA | | methyl | | Wait Time* Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

• Wait time does not include contact time during delivery.
• Tandem synthesis utilizes double coupling of linker molecule

What we claim is:

1. A short interfering RNA (siRNA) molecule that down regulates expression of a protein tyrosine phosphatase (PTP-1B) gene by RNA interference.

2. The siRNA molecule of claim 1, wherein said siRNA molecule is adapted for use to treat type I diabetes, type II diabetes, obesity or a combination thereof.

3. The siRNA molecule of claim 1, wherein said siRNA molecule comprises a sense region and an antisense region and wherein said antisense region comprises sequence complementary to an RNA sequence encoding PTP-1B and the sense region comprises sequence complementary to the antisense region.

4. The siRNA molecule of claim 3, wherein said siRNA molecule is assembled from two nucleic acid fragments wherein one fragment comprises the sense region and the second fragment comprises the antisense region of said siRNA molecule.

5. The siRNA molecule of claim 4, wherein said sense region and antisense region are covalently connected via a linker molecule.

6. The siRNA molecule of claim 5, wherein said linker molecule is a polynucleotide linker.

7. The siRNA molecule of claim 5, wherein said linker molecule is a non-nucleotide linker.

8. The siRNA molecule of claim 3, wherein said antisense region comprises sequence complementary to sequence having any of SEQ ID NOs.1-185.

9. The siRNA molecule of claim 3, wherein said antisense region comprises sequence having any of SEQ ID NOs.186-370, 384, 386, 388, 390, 392, or 394.

10. The siRNA molecule of claim 3, wherein said sense region comprises sequence having any of SEQ ID NOs.1-185, 383, 385, 387, 389, 391, or 393.

11. The siRNA molecule of claim 3, wherein said sense region comprises a sequence of SEQ ID NO.371 and said antisense region comprises a sequence of SEQ ID NO.372.

12. The siRNA molecule of claim 3, wherein said sense region comprises a sequence of SEQ ID NO.373 and said antisense region comprises a sequence of SEQ ID NO.374.

13. The siRNA molecule of claim 3, wherein said sense region comprises a sequence of SEQ ID NO.375 and said antisense region comprises a sequence of SEQ ID NO.376.

14. The siRNA molecule of claim 3, wherein said sense region comprises a sequence of SEQ ID NO.377 and said antisense region comprises a sequence of SEQ ID NO.378.

15. The siRNA molecule of claim 3, wherein said sense region comprises a sequence of SEQ ID NO.379 and said antisense region comprises a sequence of SEQ ID NO.380.

16. The siRNA molecule of claim 3, wherein said sense region comprises a sequence of SEQ ID NO.381 and said antisense region comprises a sequence of SEQ ID NO.382.

17. The siRNA molecule of claim 3, wherein said sense region comprises a 3′-terminal overhang and said antisense region comprises a 3′-terminal overhang.

18. The siRNA molecule of claim 17, wherein said 3′-terminal overhangs each comprise about 2 nucleotides.

19. The siRNA molecule of claim 17, wherein said antisense region 3′-terminal nucleotide overhang is complementary to RNA encoding PTP-1B.

20. The siRNA molecule of claim 3, wherein said sense region comprises one or more 2′-O-methyl modified pyrimidine nucleotides.

21. The siRNA molecule of claim 3, wherein said sense region comprises a terminal cap moiety at the 5′-end, 3′-end, or both 5′ and 3′ ends of said sense region.

22. The siRNA molecule of claim 3, wherein said antisense region comprises one or more 2′-deoxy-2′-fluoro modified pyrimidine nucleotides.

23. The siRNA molecule of claim 3, wherein said antisense region comprises a phosphorothioate internucleotide linkage at the 3, end of said anti sense region.

24. The siRNA molecule of claim 3, wherein said antisense region comprises between about one and about five phosphorothioate internucleotide linkages at the 5′ end of said antisense region.

25. The siRNA molecule of claim 17, wherein said 3′-terminal nucleotide overhangs comprise ribonucleotides that are chemically modified at a nucleic acid sugar, base, or backbone.

26. The siRNA molecule of claim 17, wherein said 3′-terminal nucleotide overhangs comprise deoxyribonucleotides that are modified at a nucleic acid sugar, base, or backbone.

27. The siRNA molecule of claim 17, wherein said 3′-terminal nucleotide overhangs comprise one or more universal base ribonucleotides.

28. The siRNA molecule of claim 17, wherein said 3′-terminal nucleotide overhangs comprise one or more acyclic nucleotides.

29. The siRNA molecule of claim 17, wherein said 3′-terminal nucleotide overhangs comprise nucleotides comprising internucleotide linkages having Formula I: 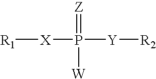
wherein each R1 and R2 is independently any nucleotide, non-nucleotide, or polynucleotide which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all O.

30. The siRNA molecule of claim 17, wherein said 3′-terminal nucleotide overhangs comprise nucleotides or non-nucleotides having Formula II: 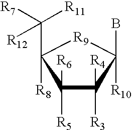
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO₂, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base or any other non-naturally occurring base that can be complementary or non-complementary to PTP-1B RNA or a non-nucleosidic base or any other non-naturally occurring universal base that can be complementary or non-complementary to PTP-1B RNA.

31. An expression vector comprising a nucleic acid sequence encoding at least one siRNA molecule of claim 1 in a manner that allows expression of the nucleic acid molecule.

32. A mammalian cell comprising an expression vector of claim 31.

33. The mammalian cell of claim 32, wherein said mammalian cell is a human cell.

34. The expression vector of claim 31, wherein said siRNA molecule comprises a sense region and an antisense region and wherein said antisense region comprises sequence complementary to an RNA sequence encoding PTP-1B and the sense region comprises sequence complementary to the antisense region.

35. The expression vector of claim 34, wherein said siRNA molecule comprises two distinct strands having complementarity sense and antisense regions.

36. The expression vector of claim 34, wherein said siRNA molecule comprises a single strand having complementary sense and antisense regions.
